(19) United States
(12) Patent Application Publication
(10) Pub. No.: US 2011/0110942 A1
(43) Pub. Date: May 12, 2011

(54) METHOD OF PROMOTING DENDRITIC SPINE DENSITY

(75) Inventors: DARA KALLOP, SAN FRANCISCO, CA (US); ANATOLY NIKOLAEV, BURLINGAME, CA (US); MARC TESSIER-LAVIGNE, ATHERTON, CA (US); ROBBY WEIMER, HALF MOON BAY, CA (US)

(73) Assignee: GENENTECH, INC., SOUTH SAN FRANCISCO, CA (US)

(21) Appl. No.: 12/944,869

(22) Filed: Nov. 12, 2010

Related U.S. Application Data
(60) Provisional application No. 61/294,020, filed on Jan. 11, 2010.
(60) Provisional application No. 61/260,815, filed on Nov. 12, 2009.

(51) Int. Cl. A61K 39/395; C07K 16/28; A61P 25/00

(52) U.S. Cl. 424/133.1; 424/158.1; 530/389.1; 530/389.2

(57) ABSTRACT

The invention relates to methods of increasing density of dendritic spines as a means to retain or improve cognition and to treat disorders associated with decreased dendritic spine morphology and a psychiatric disorder such as addiction and schizophrenia or a disorder associated with impaired cognition such as autism, Lett Syndrome, Tourette Syndrome, and Fragile-X Syndrome.

### CROSS REFERENCE TO RELATED APPLICATIONS

This application claims benefit of U.S. Provisional Application No. 61/260,815, filed Nov. 12, 2009 and U.S. Provisional Application No. 61/294,020, filed Jan. 11, 2010, the disclosure of each of which is hereby incorporated by reference in its entirety.

### FIELD OF THE INVENTION

The invention relates to a method of promoting dendritic spine density in neurons. More specifically, the invention is drawn to increasing synapses by inhibiting DR6 and/or p75 and methods of treatment of cognitive disorders.

### BACKGROUND OF THE INVENTION

The TNFR family member called DR6 receptor (also referred to in literature as “TR9”; also known in literature as TNF Receptor Superfamily Member 21 or TNFRSF21) has been described as a type I transmembrane receptor having four extracellular cysteine-rich motifs and a cytoplasmic death domain structure (Pan et al., FEBS Lett., 431:351-356 (1998); see also U.S. Pat. Nos. 6,358,508; 6,667,390; 6,919,078; 6,949,358). It has been reported that overexpression of DR6 in certain transfected cell lines resulted in apoptosis and activation of both NF-kB and JNK (Pan et al., FEBS Letters, 431:351-356 (1998)).

In a DR6-deficient mouse model, T cells were substantially impaired in JNK activation, and when DR6(−/−) mice were challenged with protein antigen, their T cells were found to hyperproliferate and display a profound polarization toward a Th2 response (whereas Th1 differentiation was not equivalently affected) (Zhao et al., J. Exp. Med., 194:1441-1448 (2001)). It was further reported that targeted disruption of DR6 resulted in enhanced T helper 2 (Th2) differentiation in vitro (Zhao et al., supra). Various uses of DR6 agonists or antagonists in modulating B-cell mediated conditions were described in US 2005/0069540 published Mar. 31, 2005. The DR6 receptor may play a role in regulating airway inflammation in the OVA-induced mouse model of asthma (Venkataraman et al., Immunol. Lett., 106:42-47 (2006)). Using a myelin oligodendrocyte glycoprotein (MOG(35-55))-induced model of experimental autoimmune encephalomyelitis, DR6−/− mice were found to be highly resistant to both the onset and the progression of CNS disease compared with wild-type (WT) littermates. Thus, DR6 may be involved in regulating leukocyte infiltration and function in the induction and progression of experimental autoimmune encephalomyelitis (Schmidt et al., J. Immunol., 175:2286-2292 (2005)).

While various TNF ligand and receptor family members have been identified as having diverse biological activities and properties, few such ligands and receptors have been reported to be involved in neurological-related functions. For example, WO2004/071528 published Aug. 26, 2004 describes inhibition of the CD95 (Fas) ligand/receptor complex in a murine model to treat spinal cord injury. Recently, Nikolaev et al. showed that an N-terminal fragment of APP is a ligand for DR6 (Nilolaev et al. (2009) Nature 457:981-989).

Nerve cells communicate with one another at synapses which on dendrites occur at “dendritic spines.” A dendritic spine is a membranous region of a dendrite that protrudes from the dendrite and is in contact with (generally) a single synapse of an axon. There may be many thousands of spines on a single dendrite. Spines receive both excitatory and inhibitory input from axons, however, excitatory input is more common. Near the tip of the dendritic spine is an electron dense region termed the post-synaptic density region (PSD). Within this region is a structural protein called PSD-95, which is a marker for the PSD. The spines are rich in glutamate receptors (e.g., AMPA and NMDA receptors). Other receptors, such as the TrkB receptor is believed to play some role in spine survival.

Chemical synapses connect neurons to form functional circuits capable of processing and storing information. The loss of proper function or stability of these connections is thought to underlie many psychiatric and neurodegenerative diseases.

### SUMMARY OF THE INVENTION

The invention provides a method of increasing density of dendritic spines in a patient with a cognitive or psychiatric disorder comprising administering to the patient an effective amount of a DR6 inhibitor and/or a p75 inhibitor. The inhibitor may be, for example, an antibody that binds to an epitope of DR6 and inhibits the function of DR6, or an antibody that binds to an epitope of p75 and inhibits the function of p75. Examples of inhibitory anti-DR6 antibodies include, but are not limited to 3F4.4.8, 4B6.9.7, 1E5.5.7, and antigen-binding fragments thereof. As such, the antibodies may be chimeric or humanized antibodies, such as, for example chimeric or humanized 3F4.4.8, 4B6.9.7 or 1E5.5.7 or an antibody that binds to the same epitope as 3F4.4.8, 4B6.9.7, or 1E5.5.7. The inhibitors of DR6 decrease or prevent DR6 signaling in neurons.

The invention also provides a method of treating a cognitive or psychiatric disorder in a patient in need thereof comprising identifying a patient having a cognitive or psychiatric disorder associated with a decrease in dentritic spines and administering to the patient a therapeutically effective amount of a DR6 antagonist and/or a p75 antagonist. The psychiatric or cognitive disorder may be, for example, Rett Syndrome, Tourette syndrome, autism, schizophrenia, or fragile-X mental retardation. The inhibitor may be, for example, an antibody that binds to an epitope of DR6 and inhibits the function of DR6, and/or an antibody that binds to an epitope of p75 and inhibits the function of p75. The antibody may be, for example, 3F4.4.8, 4B6.9.7, 1E5.5.7, or antigen-binding fragments thereof. The antibody may be a chimeric or humanized antibody, such as, for example, a chimeric or humanized 3F4.4.8, 4B6.9.7 or 1E5.5.7, or an antibody that binds to the same epitope as 3F4.4.8, 4B6.9.7, or 1E5.5.7.

The invention further provides a method of maintaining cognition in a subject during the aging process comprising administering to the subject an amount of a DR6 and/or p75 inhibitor effective to promote density of dendritic spines in the subject, thereby maintaining cognition in said subject. The inhibitor may be, for example, an antibody that binds to an epitope of DR6 and inhibits the function of DR6, and/or an antibody that binds to an epitope of p75 and inhibits the function of p75. The antibody may be, for example, 3F4.4.8, 4B6.9.7, 1E5.5.7, or antigen-binding fragments thereof. The antibody may be a chimeric or humanized antibody, such as, for example, a chimeric or humanized 3F4.4.8, 4B6.9.7 or 1E5.5.7, or an antibody that binds to the same epitope as 3F4.4.8, 4B6.9.7, or 1E5.5.7.

The invention thus provides a use of a DR6 antagonist and/or a p75 antagonist in preparation of a medicament to increase dendritic spine density and to treat patients having a cognitive or psychiatric disorder associated with decreased dendritic spine density.

### BRIEF DESCRIPTION OF THE DRAWINGS

FIG. 1 shows targeted labeling of cortical layer 2/3 excitatory neurons by in utero electroporation. Panel A: E16 embryos from pregnant mice were exposed and injected with ˜1 μl of DNA into the right lateral ventricle and an electric potential was applied; Panel B: Layer 2/3 excitatory neuron cell bodies and their processes can be seen by histology. Neurons were co-labeled with DsRedExpress and PSD-95-paGFP; Panel C: implanted cranial window; Panel D: photon microscope images (day 14 and day 44) through cranial window.

FIG. 2 shows increased density and width of dendritic spines in DR6^{-/-} animals postnatal day 60 as compared to control animals (Panel A). Density was calculated by averaging the total number of spines/dendrite length per cell across all animals within the same group. A total of 28 cells/8 animals were annotated for DR6−/− compared to 26 cells/7 animals for DR6+/− and 26 cells/6 animals for DR6+/+ (Panel B). Spine width and length plotted as a cumulative plot of the entire population of spines analyzed per genotype (Panel C).

FIG. 3 shows E16 cortical neurons in culture after treatment with 0 μg/ml N-APP (control) (Panels A and B); 1 μg/ml N-APP (Panel C); 3 μg/ml N-APP (Panel D); 10 μg/ml N-APP (Panel E); and 30 μg/ml N-APP (Panel F).

FIG. 4 shows a reduction in PSD95 puncta as a result of treatment (as compared to control) with 1, 3, 10, and 30 μg/ml N-APP.

FIG. 5 shows that N-APP-induced reduction of PSD95 puncta density is dependant on DR6 function. Percent of control (puncta per 100 um) in untreated neurons upon addition of 0.1, 0.3, 1.0, or 3.0 ug/ml N-APP (without acidic tail) or full-length N-APP (N-APP FL). One group was additionally treated with 30 ug/ml anti-DR6.1 antibody (as shown).

### DETAILED DESCRIPTION OF THE INVENTION

The techniques and procedures described or referenced herein are generally well understood and commonly employed using conventional methodology by those skilled in the art, such as, for example, the widely utilized molecular cloning methodologies described in Sambrook et al., MOLECULAR CLONING: A LABORATORY MANUAL 2ND. EDITION (1989) Cold Spring Harbor Laboratory Press, Cold Spring Harbor, N.Y. As appropriate, procedures involving the use of commercially available kits and reagents are generally carried out in accordance with manufacturer defined protocols and/or parameters unless otherwise noted.

Before the present methods and assays are described, it is to be understood that this invention is not limited to the particular methodology, protocols, cell lines, animal species or genera, constructs, and reagents described as such may, of course, vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “and,” and “the” include plural referents unless the context clearly dictates otherwise. Thus, for example, reference to “a genetic alteration” includes a plurality of such alterations and reference to “a probe” includes reference to one or more probes and equivalents thereof known to those skilled in the art, and so forth. All numbers recited in the specification and associated claims (e.g. amino acids 22-81, 1-354 etc.) are understood to be modified by the term “about.”

All publications mentioned herein are incorporated herein by reference to disclose and describe the methods and/or materials in connection with which the publications are cited. Publications cited herein are cited for their disclosure prior to the filing date of the present application. Nothing here is to be construed as an admission that the inventors are not entitled to antedate the publications by virtue of an earlier priority date or prior date of invention. Further the actual publication dates may be different from those shown and require independent verification.

The terms “Amyloid Precursor Protein” or “APP” include the various polypeptide isoforms encoded by the APP pre-mRNA, for example the APP695, APP751 and App770 isoforms shown in SEQ ID NOs: 3-5, respectively (isoforms which are translated from alternatively spliced transcripts of the APP pre-mRNA), as well as post-translationally processed portions of APP isoforms. As is known in the art, the APP pre-mRNA transcribed from the APP gene undergoes alternative exon splicing to yield a number of isoforms (see, e.g. Sandbrink et al., Ann NY Acad. Sci. 777: 281-287 (1996); and the information associated with PubMed NCBI protein locus accession P05067). This alternative exon splicing yields three major isoforms of 695 (SEQ ID NO:3), 751 SEQ ID NO:4), and 770 SEQ ID NO:5) amino acids (see, e.g. Kang et al., Nature 325: 733-736 (1987); Kitaguchi et al., Nature 331: 530-532 (1988); Ponte et al., Nature 331: 525-527 (1988); and Tanzi et al., Nature 331: 528-532 (1988)). Two of these isoforms (App₇₅₁ and APP₇₇₀) contain a 56 residue insert which is highly homologous to the Kunitz family of serine protease inhibitors (KPI) and are expressed ubiquitously. In contrast, the shorter isoform lacking the KPI motif, APP₆₉₅ is expressed predominantly in the nervous system, for example in neurons and glial cells and for this reason is often termed “neuronal APP” (see, e.g. Tanzi et al., Science 235: 880-884 (1988); Neve et al., Neuron 1: 669-677 (1988); and Haas et al., J. Neurosci. 11: 3783-3793 (1991)). The APP isoforms including the 695, 751 and 770 undergo significant post-translational processing events (see, e.g. Esch et al. 1990 Science 248:1122-1124; Sisodia et al. 1990 Science 248:492-495). For example, each of these isoforms is cleaved by various secretases and/or secretase complexes, events which produce APP fragments including a N-terminal secreted polypeptides containing the APP ectodomain (sAPPα and sAPPβ). Cleavage by alpha-secretases or alternatively by beta-secretases leads to generation and extracellular release of soluble N-terminal APP polypeptides, sAPPα and sAPPβ, respectively, and the retention of corresponding membrane-anchored C-terminal fragments, C83 and C99.

Subsequent processing of C83 by gamma-secretase yields P3 polypeptides. This is the major secretory pathway and is non-amyloidogenic. Alternatively, presenilin/nicastrin-mediated gamma-secretase processing of C99 releases the amyloid beta polypeptides, amyloid-beta 40 (Abeta40) and amyloid-beta 42 (Abeta42), major components of amyloid plaques, and the cytotoxic C-terminal fragments, gamma-CTF(50), gamma-CTF(57) and gamma-CTF(59). Evidence suggests that the relative importance of each cleavage event depends on the cell type. For example, non-neuronal cells preferentially process APP by α-secretase pathway(s) which cleaves APP within the Abeta sequence, thereby precluding the formation of Abeta (see, e.g. Esch et al. 1990 Science 248:1122-1124; Sisodia et al. 1990 Science 248:492-495). In contrast, neuronal cells process a much larger portion of APP₆₉₅ by β-secretase pathway(s), which generates intact Abeta by the combined activity of at least two enzyme classes. In neuronal cells the β-secretase(s) cleaves APP₆₉₅ at the amino terminus of the Abeta domain releasing a distinct N-terminal fragment (sAPPβ). In addition, γ-secretase(s) cleaves APP at alternative sites of the carboxy terminus generating species of Abeta that are either 40 (Abeta₄₀) or 42 amino acids long (Abeta₄₂) (see, e.g. Seubert et al. 1993 Nature 361:260-263; Suzuki et al. 1994 Science 264:1336-1340; and Turner et al. 1996 J. Biol. Chem. 271:8966-8970). It is believed that trophic deprivation triggers BACE cleavage of APP to yield the ˜100 kDa sAPPβ, which undergoes an additional cleavage(s) to yield a ˜55 kDa carboxy-terminal fragment (detected by anti-sAPPβ antibodies) and an amino-terminal ˜35 kDa fragment (detected by anti-N-APP (polyclonal antibody)), which we term “N-APP.” The site of additional cleavage(s) is unknown, but based on fragment sizes is expected to be around the junction between the APP “acidic” and “E2” domains (amino acid 286); indeed, recombinant APP[1-286] ran at ˜35 kDa and was detected with anti-N-APP(poly), similar to N-APP.

The terms “APP,” “APP protein” and “APP polypeptide” when used herein encompasses native APP sequences and APP variants and processed fragments thereof. These terms encompass APP expressed in a variety of mammals, including humans. APP may be endogenously expressed as occurs naturally in a variety of human tissue lineages, or may be expressed by recombinant or synthetic methods. A “native sequence APP” comprises a polypeptide having the same amino acid sequence as an APP derived from nature (e.g. the 695, 751 and 770 isoforms or processed portions thereof). Thus, a native sequence APP can have the amino acid sequence of naturally occurring APP from any mammal, including humans. Such native sequence APP can be isolated from nature or can be produced by recombinant or synthetic means. The term “native sequence APP” specifically encompasses naturally occurring processed and/or secreted forms of the (e.g., a soluble form containing, for instance, an extracellular domain sequence), naturally occurring variant forms (e.g., alternatively spliced and/or proteolytically processed forms) and naturally occurring allelic variants. APP variants may include fragments or deletion mutants of the native sequence APP.

APP polypeptides useful in embodiments of the invention include those described above and the following non-limiting examples. These illustrative forms can be selected for use in various embodiments of the invention. In some embodiments of the invention, the APP polypeptide comprises a full length APP isoform such as the APP₆₉₅ and/or APP₇₅₁ and/or APP₇₇₀ isoforms shown in SEQ ID NOs:3-5, respectively. In other embodiments of the invention, the APP polypeptide comprises a post-translationally processed isoform of APP, for example an APP polypeptide that has undergone cleavage by a secretase such as an α-secretase, a β-secretase or a γ-secretase (e.g. a soluble N-terminal fragment such as a sAPPα or a sAPPβ. In related embodiments of the invention, the APP polypeptide can be selected to comprise one or more specific domains such as an N-terminal ectodomain, (see, e.g. Quast et al., FASEB J. 2003; 17(12):1739-41), a heparin binding domain (see, e.g. Rossjohn et al., Nat. Struct. Biol. 1999 April; 6(4):327-31), a copper type II (see, e.g. Hesse et al., FEBS Letters 349(1): 109-116 (1994)) or a Kunitz protease inhibitor domain (see, e.g. Ponte et al., Nature; 331(6156):525-7 (1988)). In some embodiments of the invention, the APP polypeptide includes a sequence observed to comprise an epitope recognized by a DR6 antagonist disclosed herein such as an antibody or DR6 immunoadhesin, for example amino acids 22-81 of APP₆₉₅, a sequence comprising the epitope bound by monoclonal antibody 22C11 (see, e.g. Hilbich et al., J. Biol. Chem. 268(35): 26571-26577 (1993)).

In certain embodiments of the invention, the APP polypeptide does not comprise one or more specific domains or sequences, for example, an APP polypeptide that does not include the Kunitz protease inhibitor domain (e.g. APP₆₉₅), or an APP polypeptide that does not include Alzheimer's beta amyloid protein (Abeta) sequences (e.g. sAPPβ, a polypeptide which does not include the Aβ₄₀ and/or Aβ₄₂ sequences) (see, e.g. Bond et al., J. Struct Biol. 2003 February; 141(2):156-70). In other embodiments of the invention, an APP polypeptide used in embodiments of the invention comprises one or more domains or sequences but not other domains or sequences, for example an APP polypeptide that comprises an N-terminal ectodomain (or at least a portion thereof observed to be bound by a DR6 antagonist such as monoclonal antibody 22C11) but not a domain or sequence that is C-terminal to one or more secretase cleavage sites such as a beta amyloid (Abeta) sequence (e.g. a sAPPα or a sAPPβ).

The term “extracellular domain,” “ectodomain,” or “ECD” refers to a form of APP, which is essentially free of transmembrane and cytoplasmic domains. Ordinarily, the soluble ECD will have less than 1% of such transmembrane and cytoplasmic domains, and preferably, will have less than 0.5% of such domains. It will be understood that any transmembrane domain(s) identified for the polypeptides of the present invention are identified pursuant to criteria routinely employed in the art for identifying that type of hydrophobic domain. The exact boundaries of a transmembrane domain may vary but most likely by no more than about 5 amino acids at either end of the domain as initially identified. In preferred embodiments, the ECD will consist of a soluble, extracellular domain sequence of the polypeptide which is free of the transmembrane and cytoplasmic or intracellular domains (and is not membrane bound).

The term “APP variant” means a APP polypeptide as defined below having at least about 80%, preferably at least about 85%, 86%, 87%, 88%, 89%, more preferably at least about 90%, 91%, 92%, 93%, 94%, most preferably at least about 95%, 96%, 97%, 98%, or 99% amino acid sequence identity with a human APP having an amino acid sequence shown in SEQ ID NOs:3, 4, or 5 or a soluble fragment thereof, or a soluble extracellular domain thereof. Such variants include, for instance, APP polypeptides wherein one or more amino acid residues are added to, or deleted from, the N- or C-terminus of the full-length or mature sequences of APP, or APP polypeptides wherein one or more amino acid residues are inserted or deleted from the internal sequence or domains of the polypeptide, including variants from other species, but excludes a native-sequence APP polypeptide.

“DR6” or “DR6 receptor” includes the receptors referred to in the art whose polynucleotide and polypeptide sequences are known. Pan et al. have described the polynucleotide and polypeptide sequences for the TNF receptor family member referred to as “DR6” or “TR9” (Pan et al., FEBS Lett., 431:351-356 (1998); see also U.S. Pat. Nos. 6,358,508; 6,667,390; 6,919,078; 6,949,358). The human DR6 receptor is a 655 amino acid protein (SEQ ID NO:1) having a putative signal sequence (amino acids 1-41), an extracellular domain (amino acids 42-349), a transmembrane domain (amino acids 350-369), followed by a cytoplasmic domain (amino acids 370-655). The cDNA sequence for DR6 is provided as SEQ ID NO:2. The term “DR6 receptor” when used herein encompasses native sequence receptor and receptor variants. These terms encompass DR6 receptor expressed in a variety of mammals, including humans. DR6 receptor may be endogenously expressed as occurs naturally in a variety of human tissue lineages, or may be expressed by recombinant or synthetic methods. A “native sequence DR6 receptor” comprises a polypeptide having the same amino acid sequence as a DR6 receptor derived from nature. Thus, a native sequence DR6 receptor can have the amino acid sequence of naturally occurring DR6 receptor from any mammal, including humans. Such native sequence DR6 receptor can be isolated from nature or can be produced by recombinant or synthetic means. The term “native sequence DR6 receptor” specifically encompasses naturally occurring truncated or secreted forms of the receptor (e.g., a soluble form containing, for instance, an extracellular domain sequence), naturally occurring variant forms (e.g., alternatively spliced forms) and naturally occurring allelic variants. Receptor variants may include fragments or deletion mutants of the native sequence DR6 receptor.

The term “extracellular domain” or “ECD” refers to a form of DR6 receptor, which is essentially free of transmembrane and cytoplasmic domains. Ordinarily, the soluble ECD will have less than 1% of such transmembrane and cytoplasmic domains, and preferably, will have less than 0.5% of such domains. It will be understood that any transmembrane domain(s) identified for the polypeptides of the present invention are identified pursuant to criteria routinely employed in the art for identifying that type of hydrophobic domain. The exact boundaries of a transmembrane domain may vary but most likely by no more than about 5 amino acids at either end of the domain as initially identified. In preferred embodiments, the ECD will consist of a soluble, extracellular domain sequence of the polypeptide which is free of the transmembrane and cytoplasmic or intracellular domains (and is not membrane bound).

The term “DR6 variant” means a DR6 polypeptide as defined below having at least about 80%, preferably at least about 85%, 86%, 87%, 88%, 89%, more preferably at least about 90%, 91%, 92%, 93%, 94%, most preferably at least about 95%, 96%, 97%, 98%, or 99% amino acid sequence identity with human DR6 having the deduced amino acid sequence shown in SEQ ID NO:1, or a soluble fragment thereof, or a soluble extracellular domain thereof. Such variants include, for instance, DR6 polypeptides wherein one or more amino acid residues are added to, or deleted from, the N- or C-terminus of the full-length or mature sequences of SEQ ID NO:1, or DR6 polypeptides wherein one or more amino acid residues are inserted or deleted from the internal sequence or domains of the polypeptide, including variants from other species, but excludes a native-sequence DR6 polypeptide. Optionally, the DR6 variant comprises a soluble form of the DR6 receptor comprising amino acids 1-349 or 42-349 of SEQ ID NO:1 with up to 10 conservative amino acid substitutions. Preferably such a variant acts as a DR6 antagonist, as defined below.

The term “DR6 antagonist” is used in the broadest sense, and includes any molecule that partially or fully blocks, inhibits, or neutralizes the ability of DR6 receptor to bind its cognate ligand, preferably, its cognate ligand APP, or to activate one or more intracellular signal(s) or intracellular signaling pathway(s) in neuronal cells or tissue, either in vitro, in situ, in vivo or ex vivo. By way of example, a DR6 antagonist may partially or fully block, inhibit, or neutralize the ability of DR6 receptor to activate one or more intracellular signal(s) or intracellular signaling pathway(s) in neuronal cells or tissue that results in apoptosis or cell death in the neuronal cells or tissue. The DR6 antagonist may act to partially or fully block, inhibit, or neutralize DR6 by a variety of mechanisms, including but not limited to, by blocking, inhibiting, or neutralizing binding of cognate ligand to DR6, formation of a complex between DR6 and its cognate ligand (e.g. APP), oligomerization of DR6 receptors, formation of a complex between DR6 receptor and heterologous co-receptor, binding of a cognate ligand to DR6 receptor/heterologous co-receptor complex, or formation of a complex between DR6 receptor, heterologous co-receptor, and its cognate ligand. DR6 antagonists may function in a direct or indirect manner. DR6 antagonists contemplated by the invention include but are not limited to, APP antibodies, DR6 antibodies, immunoadhesins, DR6 immunoadhesins, DR6 fusion proteins, covalently modified forms of DR6, DR6 variants and fusion proteins thereof, or higher oligomer forms of DR6 (dimers, aggregates) or homo- or heteropolymer forms of DR6, small molecules such as pharmacological inhibitors of the JNK signaling cascade, including small molecule and peptide inhibitors of Jun N-terminal kinase JNK activity, pharmacological inhibitors of protein kinases MLKs and MKKs activities that function upstream of JNK in the signal transduction pathway, pharmacological inhibitors of binding of JNK to scaffold protein JIP-1, pharmacological inhibitors of binding of JNK to its substrates such as c-Jun or AP-1 transcription factor complexes, pharmacological inhibitors of JNK-mediated phosphorylation of its substrates such as JNK binding domain (JBD) peptide and/or substrate binding domain of JNK and/or peptide inhibitor comprising JNK substrate phosphorylation site, small molecules that block ATP binding to JNK, and small molecules that block substrate binding to JNK.

To determine whether a DR6 antagonist partially or fully blocks, inhibits or neutralizes the ability of DR6 receptor to activate one or more intracellular signal(s) or intracellular signaling pathway(s) in neuronal cells or tissue, assays may be conducted to assess the effect(s) of the DR6 antagonist on, for example, various neuronal cells or tissues as well as in in vivo models. The various assays may be conducted in known in vitro or in vivo assay formats, such as described below or as known in the art and described in the literature. One embodiment of an assay to determine whether a DR6 antagonist partially or fully blocks, inhibits or neutralizes the ability of DR6 receptor to activate one or more intracellular signal(s) or intracellular signaling pathway(s) in neuronal cells or tissue, comprises combining DR6 and APP in the presence or absence of a DR6 antagonist or potential DR6 antagonist (i.e. a molecule of interest); and then detecting inhibition of binding of DR6 to APP in the presence of this DR6 antagonist or potential DR6 antagonist.

By “nucleic acid” is meant to include any DNA or RNA. For example, chromosomal, mitochondrial, viral and/or bacterial nucleic acid present in tissue sample. The term “nucleic acid” encompasses either or both strands of a double stranded nucleic acid molecule and includes any fragment or portion of an intact nucleic acid molecule.

By “gene” is meant any nucleic acid sequence or portion thereof with a functional role in encoding or transcribing a protein or regulating other gene expression. The gene may consist of all the nucleic acids responsible for encoding a functional protein or only a portion of the nucleic acids responsible for encoding or expressing a protein. The nucleic acid sequence may contain a genetic abnormality within exons, introns, initiation or termination regions, promoter sequences, other regulatory sequences or unique adjacent regions to the gene.

The terms “amino acid” and “amino acids” refer to all naturally occurring L-alpha-amino acids. This definition is meant to include norleucine, ornithine, and homocysteine. The amino acids are identified by either the single-letter or three-letter designations:

| | | | | | |
|---|---|---|---|---|---|
| Asp | D | aspartic acid | Ile | I | isoleucine |
| Thr | T | threonine | Leu | L | leucine |
| Ser | S | serine | Tyr | Y | tyrosine |
| Glu | E | glutamic acid | Phe | F | phenylalanine |
| Pro | P | proline | His | H | histidine |
| Gly | G | glycine | Lys | K | lysine |
| Ala | A | alanine | Arg | R | arginine |
| Cys | C | cysteine | Trp | W | tryptophan |
| Val | V | valine | Gln | Q | glutamine |
| Met | M | methionine | Asn | N | asparagine |

“Isolated,” when used to describe the various peptides or proteins disclosed herein, means peptide or protein that has been identified and separated and/or recovered from a component of its natural environment. Contaminant components of its natural environment are materials that would typically interfere with diagnostic or therapeutic uses for the peptide or protein, and may include enzymes, hormones, and other proteinaceous or non-proteinaceous solutes. In preferred embodiments, the peptide or protein will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain, or (3) to homogeneity by mass spectroscopic or peptide mapping techniques. Isolated material includes peptide or protein in situ within recombinant cells, since at least one component of its natural environment will not be present. Ordinarily, however, isolated peptide or protein will be prepared by at least one purification step.

“Percent (%) amino acid sequence identity” with respect to the sequences identified herein is defined as the percentage of amino acid residues in a candidate sequence that are identical with the amino acid residues in the reference sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art can determine appropriate parameters for measuring alignment, including assigning algorithms needed to achieve maximal alignment over the full-length sequences being compared. For purposes herein, percent amino acid identity values can be obtained using the sequence comparison computer program, ALIGN-2, which was authored by Genentech, Inc. and the source code of which has been filed with user documentation in the US Copyright Office, Washington, D.C., 20559, registered under the US Copyright Registration No. TXU510087. The ALIGN-2 program is publicly available through Genentech, Inc., South San Francisco, Calif. All sequence comparison parameters are set by the ALIGN-2 program and do not vary.

The term “primer” or “primers” refers to oligonucleotide sequences that hybridize to a complementary RNA or DNA target polynucleotide and serve as the starting points for the stepwise synthesis of a polynucleotide from mononucleotides by the action of a nucleotidyltransferase, as occurs for example in a polymerase chain reaction.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The word “label” when used herein refers to a compound or composition which is conjugated or fused directly or indirectly to a reagent such as a nucleic acid probe or an antibody and facilitates detection of the reagent to which it is conjugated or fused. The label may itself be detectable (e.g., radioisotope labels or fluorescent labels) or, in the case of an enzymatic label, may catalyze chemical alteration of a substrate compound or composition which is detectable.

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2, IgG-3, or IgG-4 subtypes, IgA (including IgA-1 and IgA-2), IgE, IgD or IgM.

“DR6 receptor antibody,” “DR6 antibody,” or “anti-DR6 antibody” is used in a broad sense to refer to antibodies that bind to at least one form of a DR6 receptor, preferably a human DR6 receptor, such as the DR6 sequence shown in SEQ ID NO:1 or an extracellular domain sequence thereof. Optionally the DR6 antibody is fused or linked to a heterologous sequence or molecule. Preferably the heterologous sequence allows or assists the antibody to form higher order or oligomeric complexes. The term “anti-DR6 antibody” and its grammatical equivalents specifically encompass the DR6 monoclonal antibodies described below. Optionally, the DR6 antibody binds to DR6 receptor but does not bind or cross-react with any additional receptor of the tumor necrosis factor family (e.g. DR4, DR5, TNFR1, TNFR2, Fas). Optionally, the DR6 antibody of the invention binds to a DR6 receptor at a concentration range of about 0.067 nM to about 0.033 μM as measured in a BIAcore binding assay.

The terms “anti-APP antibody,” “APP antibody” and grammatical equivalents are used in a broad sense and refer to antibodies that bind to at least one form of APP, preferably a human APP such as the APP polypeptides isoforms specifically described herein. Preferably, the APP antibody is a DR6 antagonist antibody. For example, in methods for making and/or identifying DR6 antagonists as disclosed herein, one or more isoforms of APP and/or a portion thereof can be used as an immunogen to immunize an animal (e.g. a mouse as part of a process for generating a monoclonal antibody) and/or as a probe to screen a library of compounds (e.g. a recombinant antibody library). Typical APP polypeptides useful in embodiments of the invention include the following non-limiting examples. These illustrative forms can be selected for use in various embodiments of the invention. In some embodiments of the invention, the APP polypeptide comprises a full length APP isoform such as the APP₆₉₅ and/or APP₇₅₁ and/or APP₇₇₀ isoforms shown in SEQ ID NOs: 3, 4, and 5, respectively. In other embodiments of the invention, the APP polypeptide comprises a post-translationally processed isoform of APP, for example an APP polypeptide that has undergone cleavage by a secretase such as an α-secretase, a β-secretase or a γ-secretase (e.g. a soluble N-terminal fragment such as a sAPPα or a sAPPβ). In related embodiments of the invention, the APP polypeptide can be selected to comprise one or more specific domains such as an N-terminal ectodomain, (see, e.g. Quast et al., FASEB J. 2003; 17(12):1739-41), a heparin binding domain (see, e.g. Rossjohn et al., Nat. Struct. Biol. 1999 April; 6(4):327-31), a copper type II (see, e.g. Hesse et al., FEBS Letters 349(1): 109-116 (1994)) or a Kunitz protease inhibitor domain (see, e.g. Ponte et al., Nature; 331(6156):525-7 (1988)). In some embodiments of the invention, the APP polypeptide includes a sequence observed to comprise an epitope recognized by a DR6 antagonist disclosed herein such as an antibody or DR6 immunoadhesin, for example amino acids 22-81 of APP₆₉₅, a sequence comprising the epitope bound by monoclonal antibody 22C11 (see, e.g. Hilbich et al., J. Biol. Chem., 268(35): 26571-26577 (1993)). In certain embodiments of the invention, the APP polypeptide does not comprise one or more specific domains or sequences, for example an APP polypeptide that does not include certain N-terminal or C-terminal amino acids, an APP polypeptide that does not include the Kunitz protease inhibitor domain (e.g. APP₆₉₅), or an APP polypeptide that does not include Alzheimer's beta amyloid protein (Abeta) sequences (e.g. sAPPβ, a polypeptide which does not include the Aβ₄₀ and/or Aβ₄₂ sequences) (see, e.g. Bond et al., J. Struct Biol. 2003 February; 141(2):156-70). In other embodiments of the invention, an APP polypeptide used in embodiments of the invention comprises one or more domains or sequences but not other domains or sequences, for example an APP polypeptide that comprises an N-terminal ectodomain (or at least a portion thereof observed to be bound by a DR6 antagonist such as monoclonal antibody 22C11) but not a domain or sequence that is C-terminal to one or more secretase cleavage sites such as a beta amyloid (Abeta) sequence (e.g. a sAPPα or a sAPPβ). Optionally, the anti-APP antibody will inhibit binding of the N-APP polypeptide to DR6 and bind to an N-APP polypeptide at concentrations of 10 μg/ml to 50 μg/ml, as described herein, and/or as measured in a quantitative cell-based binding assay.

The term “antibody” herein is used in the broadest sense and specifically covers intact monoclonal antibodies, polyclonal antibodies, multispecific antibodies (e.g. bispecific antibodies) formed from at least two intact antibodies, and antibody fragments so long as they exhibit the desired biological activity.

“Antibody fragments” comprise a portion of an intact antibody, preferably comprising the antigen-binding or variable region thereof. Examples of antibody fragments include Fab, Fab′, F(ab′)₂, and Fv fragments; diabodies; linear antibodies; single-chain antibody molecules; and multispecific antibodies formed from antibody fragments.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light-chain variable domain is aligned with the variable domain of the heavy chain. Particular amino acid residues are believed to form an interface between the light chain and heavy chain variable domains.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable or complementary determining regions both in the light chain and the heavy chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., SEQUENCES OF PROTEINS OF IMMUNOLOGICAL INTEREST, 5th Ed. Public Health Service, National Institutes of Health, Bethesda, Md. (1991)). The constant domains are not involved directly in binding an antibody to an antigen, but exhibit various effector functions, such as participation of the antibody in antibody-dependent cell-mediated cytotoxicity (ADCC).

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-binding sites and is still capable of cross-linking antigen.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known.

“Single-chain Fv” or “scFv” antibody fragments comprise the V_{H} and V_{L} domains of antibody, wherein these domains are present in a single polypeptide chain. Preferably, the Fv polypeptide further comprises a polypeptide linker between the V_{H} and V_{L} domains which enables the scFv to form the desired structure for antigen binding. For a review of scFv see Plückthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, such as baboon, rhesus or cynomolgus monkey) and human constant region sequences (U.S. Pat. No. 5,693,780).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

The term “hypervariable region” when used herein refers to the amino acid residues of an antibody which are responsible for antigen-binding. The hypervariable region comprises amino acid residues from a “complementarity determining region” or “CDR” (e.g. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). “Framework” or “FR” residues are those variable domain residues other than the hypervariable region residues as herein defined.

An antibody “which binds” an antigen of interest is one capable of binding that antigen with sufficient affinity and/or avidity such that the antibody is useful as a therapeutic or diagnostic agent for targeting a cell expressing the antigen.

For the purposes herein, “immunotherapy” will refer to a method of treating a mammal (preferably a human patient) with an antibody, wherein the antibody may be an unconjugated or “naked” antibody, or the antibody may be conjugated or fused with heterologous molecule(s) or agent(s), such as one or more cytotoxic agent(s), thereby generating an “immunoconjugate.”

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The term “tagged” when used herein refers to a chimeric molecule comprising an antibody or polypeptide fused to a “tag polypeptide”. The tag polypeptide has enough residues to provide an epitope against which an antibody can be made or to provide some other function, such as the ability to oligomerize (e.g. as occurs with peptides having leucine zipper domains), yet is short enough such that it generally does not interfere with activity of the antibody or polypeptide. The tag polypeptide preferably also is fairly unique so that a tag-specific antibody does not substantially cross-react with other epitopes. Suitable tag polypeptides generally have at least six amino acid residues and usually between about 8 to about 50 amino acid residues (preferably, between about 10 to about 20 residues).

The terms “Fc receptor” or “FcR” are used to describe a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see Daëron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)). FcRs herein include polymorphisms such as the genetic dimorphism in the gene that encodes FcγRIIIa resulting in either a phenylalanine (F) or a valine (V) at amino acid position 158, located in the region of the receptor that binds to IgG1. The homozygous valine FcγRIIIa (FcγRIIIa-158V) has been shown to have a higher affinity for human IgG1 and mediate increased ADCC in vitro relative to homozygous phenylalanine FcγRIIIa (FcγRIIIa-158F) or heterozygous (FcγRIIIa-158F/V) receptors.

The term “polyol” when used herein refers broadly to polyhydric alcohol compounds. Polyols can be any water-soluble poly(alkylene oxide) polymer for example, and can have a linear or branched chain. Preferred polyols include those substituted at one or more hydroxyl positions with a chemical group, such as an alkyl group having between one and four carbons. Typically, the polyol is a poly(alkylene glycol), preferably poly(ethylene glycol) (PEG). However, those skilled in the art recognize that other polyols, such as, for example, poly(propylene glycol) and polyethylene-polypropylene glycol copolymers, can be employed using the techniques for conjugation described herein for PEG. The polyols include those well known in the art and those publicly available, such as from commercially available sources such as Nektar® Corporation.

The term “conjugate” is used herein according to its broadest definition to mean joined or linked together. Molecules are “conjugated” when they act or operate as if joined.

The expression “effective amount” refers to an amount of an agent (e.g. DR6 antagonist etc.) which is effective for preventing, ameliorating or treating the disorder or condition in question. It is contemplated that the DR6 antagonists of the invention will be useful in promoting density of dendritic spines and retention of PSD-95.

The terms “treating,” “treatment” and “therapy” as used herein refer to curative therapy, prophylactic therapy, and preventative therapy. Consecutive treatment or administration refers to treatment on at least a daily basis without interruption in treatment by one or more days. Intermittent treatment or administration, or treatment or administration in an intermittent fashion, refers to treatment that is not consecutive, but rather cyclic in nature.

As used herein, the term “disorder” in general refers to any condition that would benefit from treatment with the DR6 antagonists described herein. This includes chronic and acute disorders, as well as those pathological conditions which predispose the mammal to the disorder in question.

“Neuronal cells or tissue” refers generally to motor neurons, interneurons including but not limited to commissural neurons, sensory neurons including but not limited to dorsal root ganglion neurons, dopamine (DA) neurons of substantia nigra, striatal DA neurons, cortical neurons, brainstem neurons, spinal cord interneurons and motor neurons, hippocampal neurons including but not limited to CA1 pyramidal neurons of the hippocampus, and forebrain neurons. The term neuronal cells or tissue is intended herein to refer to neuronal cells consisting of a cell body, axon(s) and dendrite(s), as well as to axon(s) or dendrite(s) that may form part of such neuronal cells.

“Psychiatric disorder” is used herein to refer to conditions that include such disorders as schizophrenia and addiction. “Cognitive disorders” include such disorders as autism, Tourette Syndrome, Rett Syndrome and Fragile-X Syndrome mental retardation.

By “subject” or “patient” is meant any single subject for which therapy is desired, including humans. Also intended to be included as a subject are any subjects involved in clinical research trials not showing any clinical sign of disease, or subjects involved in epidemiological studies, or subjects used as controls.

The term “mammal” as used herein refers to any mammal classified as a mammal, including humans, cows, horses, dogs and cats. In a preferred embodiment of the invention, the mammal is a human.

Chemical synapses connect neurons to form functional circuits capable of processing and storing information. The loss of proper function or stability of these connections is thought to underlie many psychiatric and cognitive disorders. It is believed that loss of dendritic spines or instability of dendritic spines, and alteration of dendritic spine-associated proteins such as PSD-95 is associated with such disorders as Rett Syndrome, Tourette Syndrome, schizophrenia, autism, addiction and Fragile-X Syndrome.

Applicants surprisingly found that DR6, a member of the TNFR family, is highly expressed in embryonic and adult central nervous system, including cerebral cortex, hippocampus, motor neurons and interneurons of the spinal cord. As described in the Examples below, Applicants conducted various experimental assays to examine the role of DR6 in synaptic stability in vivo.

Applicants further hypothesize that a portion of the amyloid precursor protein (N-APP) is a cognate ligand of DR6 receptor and therefore, APP also has a role in synaptic stability. In a recent paper by Bittner, et al. (Bittner, T., et al. (2009) J. Neurosci. 29(33):10405-10409), the authors showed that dendritic spine density was higher in APP mice mice than in wild type mice, and even higher in APP^{-/-} mice. Amyloid precursor protein has previously been hypothesized to play some, though not fully understood, role in Alzheimer's disease (Selkoe, J. Biol. Chem. 271:18295 (1996); Scheuner; et al., Nature Med. 2:864 (1996); Goate, et al., Nature 349:704 (1991)).

It is believed that DR6 and/or APP inhibitors will be particularly useful in treating various psychiatric and cognitive disorders. Such inhibitors may also be useful in enhancing cognition or maintaining cognition during the aging process.

The present invention accordingly provides DR6 and/or APP antagonist compositions and methods for inhibiting, blocking or neutralizing DR6 and/or APP activity in a mammal which comprise administration of an effective amount of DR6 and/or APP antagonist. Preferably, the amount of DR6 and/or APP antagonist employed will be an amount effective to promote density of dendritic spines and maintain healthy synapses. The amount of antagonist employed may also increase the expression or enhance retention of PDS-95 in dendritic spines. In some instances, it may be beneficial to employ antagonists of p75 in conjunction with or separately from DR6 and/or APP antagonists.

The DR6 antagonists which can be employed in the methods include, but are not limited to, DR6 and/or APP immunoadhesins, fusion proteins comprising DR6 and/or APP, covalently modified forms of DR6 and/or APP, DR6 and/or APP variants, fusion proteins thereof, and DR6 and/or APP antibodies. The p75 antagonists which can be employed in the methods include, but are not limited to, p75 immunoadhesins, fusion proteins comprising p75, covalently modified forms of p75, p75 variants, fusion proteins thereof, and p75 antibodies. The anti-p75 antibodies may be any known in the art. The protein sequence of p75 is provided as SEQ ID NO:6. Various techniques that can be employed for making the antagonists are described herein. For instance, methods and techniques for preparing DR6, p75 and APP polypeptides are described. Further modifications of the DR6, p75 and APP polypeptides, and antibodies to DR6, p75 and APP are also described.

The invention disclosed herein has a number of embodiments. The invention provides methods of inhibiting binding of DR6 to APP comprising exposing DR6 polypeptide and/or APP polypeptide to one or more DR6 antagonists under conditions wherein binding of DR6 to APP is inhibited. Related embodiments of the invention provide methods of inhibiting binding of DR6 polypeptide comprising amino acids 1-655 of SEQ ID NO:1 and an APP polypeptide comprising amino acids 66-81 of SEQ ID NO:3 (e.g. sAPPβ), the method comprising combining the DR6 polypeptide and the APP polypeptide with an isolated antagonist that binds DR6 or APP, wherein the isolated antagonist is chosen from at least one of an antibody that binds APP, an antibody that binds DR6 and a soluble DR6 polypeptide comprising amino acids 1-354 of SEQ ID NO:1; and the isolated antagonist is selected for its ability to inhibit binding of DR6 and APP; so that binding of DR6 to APP is inhibited.

The invention also provides methods of inhibiting binding of DR6 to APP and inhibiting the binding of p75 to APP comprising exposing DR6 polypeptide, p75 polypeptide and, optionally, APP polypeptide to one or more DR6 antagonists and one or more p75 antagonists under conditions wherein binding of DR6 and p75 to APP is inhibited. Related embodiments of the invention provide methods of inhibiting binding of DR6 polypeptide comprising amino acids 1-655 of SEQ ID NO:1 and an APP polypeptide comprising amino acids 66-81 of SEQ ID NO:3 (e.g. sAPPβ), the method comprising combining the DR6 polypeptide and the APP polypeptide with an isolated antagonist that binds DR6 or APP, and an antagonist that binds p75, wherein the isolated DR6 antagonist is chosen from at least one of an antibody that binds APP, an antibody that binds DR6 and a soluble DR6 polypeptide comprising amino acids 1-354 of SEQ ID NO:1; and the isolated DR6 antagonist is selected for its ability to inhibit binding of DR6 and APP; so that binding of DR6 to APP is inhibited. The isolated p75 antagonist is chosen from at least one of an antibody that binds p75, and a soluble p75 polypeptide comprising amino acids of the extracellular domain of p75 (e.g., amino acids 29-250 of SEQ ID NO:6); and the isolated p75 antagonist is selected for its ability to inhibit binding of p75 and APP; so that binding of p75 to APP is inhibited.

Optionally in such methods, one or more of DR6 antagonists are selected from an antibody that binds DR6 (e.g. an antibody that binds DR6 competitively inhibits binding of the 3F4.4.8, 4B6.9.7, or 1E5.5.7 monoclonal antibody produced by the hybridoma cell line deposited as ATCC accession number PTA-8095, PTA-8094, or PTA-8096, respectively), a soluble DR6 polypeptide comprising amino acids 1-354 of SEQ ID NO:1 (e.g. a DR6 immunoadhesin), or an antibody that binds APP (e.g. monoclonal antibody 22C11). In certain embodiments of the invention, a DR6 antagonist is an antibody that binds DR6, antibody that binds APP or soluble DR6 polypeptide that is linked to one or more non-proteinaceous polymers selected from the group consisting of polyethylene glycol, polypropylene glycol, and polyoxyalkylene. The p75 antagonist may also be linked to one or more non-proteinaceous polymers selected from the group consisting of polyethylene glycol, polypropylene glycol, and polyoxyalkylene.

In optional embodiments of these methods, the DR6 polypeptide, alone or in combination with p75 polypeptide, is expressed on the cell surface of one or more mammalian cells (e.g. commissural neuron cell, a sensory neuron cell or a motor neuron cell) and binding of said one or more DR6 antagonists, and/or p75 antagonists inhibits DR6 activation or signaling and/or p75 activation or signaling.

In further embodiments of the invention, methods of inhibiting binding of DR6, and optionally, p75 to APP may be conducted in vivo in a mammal having a psychiatric condition or disorder or a cognitive disorder. Optionally, the psychiatric condition or disorder is schizophrenia or addiction. Alternatively, the cognitive condition or disorder comprises Tourette Syndrome, Lett Syndrome, Fragile-X Syndrome, or autism. Further embodiments of the invention provide methods of treating a mammal having a condition or disorder, comprising administering to said mammal an effective amount of one or more DR6 antagonists, alone or in combination with one or more p75 antagonists. Typically in such methods, the one or more DR6 antagonists are selected from an antibody that binds DR6, a soluble DR6 polypeptide comprising amino acids 1-354 of SEQ ID NO:1, a DR6 immunoadhesin, and an antibody that binds APP. The one or more p75 antagonists are selected from an antibody that binds p75, a p75 immunoadhesin, and a soluble p75 polypeptide comprising amino acids 29-250 of SEQ ID NO:6. In optional embodiments of the invention, the condition or disorder is autism, Fragile-X Syndrome, Lett Syndrome, Tourette Syndrome, addiction and schizophrenia. In various embodiments of the invention, one or more further therapeutic agents is administered to said mammal. In certain illustrative embodiments of the invention, the one or more further therapeutic agents are selected from NGF, an apoptosis inhibitor, an EGFR inhibitor, a β-secretase inhibitor, a γ-secretase inhibitor, a cholinesterase inhibitor, an anti-Abeta antibody and a NMDA receptor antagonist. Optionally, the one or more DR6 antagonists, p75 antagonists and/or further therapeutic agents is administered to the mammal via injection, infusion or perfusion.

In addition to the full-length native sequence DR6, p75 and APP polypeptides described herein, it is contemplated that DR6, p75 and APP polypeptide variants can be prepared. DR6, p75 and/or APP variants can be prepared by introducing appropriate nucleotide changes into the encoding DNA, and/or by synthesis of the desired polypeptide. Those skilled in the art will appreciate that amino acid changes may alter post-translational processes of the DR6, p75 and/or APP polypeptide, such as changing the number or position of glycosylation sites or altering the membrane anchoring characteristics.

Variations in the DR6, p75 and/or APP polypeptides described herein, can be made, for example, using any of the techniques and guidelines for conservative and non-conservative mutations set forth, for instance, in U.S. Pat. No. 5,364,934. Variations may be a substitution, deletion or insertion of one or more codons encoding the polypeptide that results in a change in the amino acid sequence as compared with the native sequence polypeptide. Optionally the variation is by substitution of at least one amino acid with any other amino acid in one or more of the domains of the DR6, p75 and/or APP polypeptide. Guidance in determining which amino acid residue may be inserted, substituted or deleted without adversely affecting the desired activity may be found by comparing the sequence of the DR6, p75 and/or APP polypeptides with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 5 amino acids. The variation allowed may be determined by systematically making insertions, deletions or substitutions of amino acids in the sequence and testing the resulting variants for DR6, p75 and/or APP antagonistic activity.

DR6, p75 and/or APP polypeptide fragments are provided herein. Such fragments may be truncated at the N-terminus or C-terminus, or may lack internal residues, for example, when compared with a full length native protein. Certain fragments lack amino acid residues that are not essential for the desired biological activity of the DR6 polypeptide.

DR6, p75 and/or APP polypeptide fragments may be prepared by any of a number of conventional techniques. Desired peptide fragments may be chemically synthesized. An alternative approach involves generating polypeptide fragments by enzymatic digestion, e.g., by treating the protein with an enzyme known to cleave proteins at sites defined by particular amino acid residues, or by digesting the DNA with suitable restriction enzymes and isolating the desired fragment. Yet another suitable technique involves isolating and amplifying a DNA fragment encoding a desired polypeptide fragment, by polymerase chain reaction (PCR). Oligonucleotides that define the desired termini of the DNA fragment are employed at the 5′ and 3′ primers in the PCR.

In particular embodiments, conservative substitutions of interest are shown in the Table below under the heading of preferred substitutions. If such substitutions result in a change in biological activity, then more substantial changes, denominated exemplary substitutions in the Table, or as further described below in reference to amino acid classes, are introduced and the products screened.

| Original Residue | Exemplary Substitutions | Preferred Substitutions |
|---|---|---|
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; phe; | leu |
| | norleucine | |
| Leu (L) | norleucine; ile; val; met; ala; | ile |
| | phe | |
| Lys (K) | arg; gln; asn | arg |
| Met (M) | leu; phe; ile | leu |
| Phe (F) | leu; val; ile; ala; tyr | leu |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; ala; | leu |
| | norleucine | |

Substantial modifications in function or immunological identity of the DR6, p75 and/or APP polypeptides are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gln, his, lys, arg;

(5) residues that influence chain orientation: gly, pro; and

(6) aromatic: trp, tyr, phe.

Non-conservative substitutions will entail exchanging a member of one of these classes for another class. Such substituted residues also may be introduced into the conservative substitution sites or, more preferably, into the remaining (non-conserved) sites.

The variations can be made using methods known in the art such as oligonucleotide-mediated (site-directed) mutagenesis, alanine scanning, and PCR mutagenesis. Site-directed mutagenesis (Carter et al., Nucl. Acids Res., 13:4331 (1986); Zoller et al., Nucl. Acids Res., 10:6487 (1987)), cassette mutagenesis (Wells et al., Gene, 34:315 (1985)), restriction selection mutagenesis (Wells et al., Philos. Trans. R. Soc. London SerA, 317:415 (1986)) or other known techniques can be performed on the cloned DNA to produce the DR6 polypeptide variant DNA.

Scanning amino acid analysis can also be employed to identify one or more amino acids along a contiguous sequence. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant (Cunningham and Wells, Science, 244:1081-1085 (1989)). Alanine is also typically preferred because it is the most common amino acid. Further, it is frequently found in both buried and exposed positions (Creighton, THE PROTEINS, (W.H. Freeman & Co., N.Y.); Chothia, J. Mol. Biol., 150:1 (1976)). If alanine substitution does not yield adequate amounts of variant, an isoteric amino acid can be used.

Any cysteine residue not involved in maintaining the proper conformation of the DR6, p75 and/or APP polypeptide also may be substituted, generally with serine, to improve the oxidative stability of the molecule and prevent aberrant crosslinking Conversely, cysteine bond(s) may be added to the DR6, p75 and/or APP polypeptide to improve its stability.

Embodiments of the invention disclosed herein apply to a wide variety of APP polypeptides. In certain embodiments of the invention for example, an APP is the full length 695, 750 or 770 APP isoform shown in SEQ ID NOs:3-5, respectively. In other embodiments of the invention, the APP comprises an n-terminal portion of APP having the APP ectodomain and which is which produced from a post-translational processing event (e.g. sAPPα or sAPPβ). Optionally for example, an APP can comprise a soluble form of one of 695, 750 or 770 APP isoforms that results from cleavage by a secretase, for example a soluble form of neuronal APP₆₉₅ that results from cleavage by a β-secretase. In a specific illustrative embodiment, an APP comprises amino acids 20-591 of APP₆₉₅ (see, e.g. Jin et al., J. Neurosci., 14(9): 5461-5470 (1994). In another embodiment of the invention, an APP comprises a polypeptide having the epitope recognized by monoclonal antibody 22C11 (e.g. as is available from Chemicon International Inc., Temecula, Calif., U.S.A.). Optionally, an APP comprises residues 66-81 of APP₆₉₅, a region containing the 22C11 epitope (see, e.g. Hilbrich, J. Biol. Chem. 268 (35):26571-26577 (1993)).

The description below relates primarily to production of DR6, p75 and/or APP polypeptides by culturing cells transformed or transfected with a vector containing a DR6, p75 and/or APP polypeptide-encoding nucleic acid. It is, of course, contemplated that alternative methods, which are well known in the art, may be employed to prepare DR6, p75 and/or APP polypeptides. For instance, the appropriate amino acid sequence, or portions thereof, may be produced by direct peptide synthesis using solid-phase techniques (see, e.g., Stewart et al., SOLID-PHASE PEPTIDE SYNTHESIS, W.H. Freeman Co., San Francisco, Calif. (1969); Merrifield, J. Am. Chem. Soc., 85:2149-2154 (1963)). In vitro protein synthesis may be performed using manual techniques or by automation. Automated synthesis may be accomplished, for instance, using an Applied Biosystems Peptide Synthesizer (Foster City, Calif.) using manufacturer's instructions. Various portions of the DR6 and/or APP polypeptide may be chemically synthesized separately and combined using chemical or enzymatic methods to produce the desired DR6, p75 and/or APP polypeptide.

The methods and techniques described are similarly applicable to production of DR6, p75 and/or APP variants, modified forms of DR6, p75 and/or APP; and DR6, p75 and/or APP antibodies.

Isolation of DNA Encoding DR6 and/or APP Polypeptides

DNA encoding DR6, p75 and/or APP polypeptide may be obtained from a cDNA library prepared from tissue believed to possess the DR6, p75 and/or APP polypeptide mRNA and to express it at a detectable level. Accordingly, human DR6, p75 and/or APP polypeptide DNA can be conveniently obtained from a cDNA library prepared from human tissue. The DR6, p75 and/or APP polypeptide-encoding gene may also be obtained from a genomic library or by known synthetic procedures (e.g., automated nucleic acid synthesis).

Libraries can be screened with probes (such as oligonucleotides of at least about 20-80 bases) designed to identify the gene of interest or the protein encoded by it. Screening the cDNA or genomic library with the selected probe may be conducted using standard procedures, such as described in Sambrook et al., MOLECULAR CLONING: A LABORATORY MANUAL (New York: Cold Spring Harbor Laboratory Press, 1989). An alternative means to isolate the gene encoding DR6 polypeptide is to use PCR methodology (Sambrook et al., supra; Dieffenbach et al., PCR PRIMER: A LABORATORY MANUAL (Cold Spring Harbor Laboratory Press, 1995)).

Techniques for screening a cDNA library are well known in the art. The oligonucleotide sequences selected as probes should be of sufficient length and sufficiently unambiguous that false positives are minimized. The oligonucleotide is preferably labeled such that it can be detected upon hybridization to DNA in the library being screened. Methods of labeling are well known in the art, and include the use of radiolabels like ³²P-labeled ATP, biotinylation or enzyme labeling. Hybridization conditions, including moderate stringency and high stringency, are provided in Sambrook et al., supra.

Sequences identified in such library screening methods can be compared and aligned to other known sequences deposited and available in public databases such as GenBank or other private sequence databases. Sequence identity (at either the amino acid or nucleotide level) within defined regions of the molecule or across the full-length sequence can be determined using methods known in the art and as described herein.

Nucleic acid having protein coding sequence may be obtained by screening selected cDNA or genomic libraries using the deduced amino acid sequence disclosed herein for the first time, and, if necessary, using conventional primer extension procedures as described in Sambrook et al., supra, to detect precursors and processing intermediates of mRNA that may not have been reverse-transcribed into cDNA.

### Selection and Transformation of Host Cells

Host cells are transfected or transformed with expression or cloning vectors described herein for DR6, p75 and/or APP polypeptide production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in MAMMALIAN CELL BIOTECHNOLOGY: A PRACTICAL APPROACH, M. Butler, ed. (IRL Press, 1991) and Sambrook et al., supra.

Methods of eukaryotic cell transfection and prokaryotic cell transformation are known to the ordinarily skilled artisan, for example, CaCl₂, CaPO₄, liposome-mediated and electroporation. Depending on the host cell used, transformation is performed using standard techniques appropriate to such cells. The calcium treatment employing calcium chloride, as described in Sambrook et al., supra, or electroporation is generally used for prokaryotes. Infection with Agrobacterium tumefaciens is used for transformation of certain plant cells, as described by Shaw et al., Gene, 23:315 (1983) and WO 89/05859 published 29 Jun. 1989. For mammalian cells without such cell walls, the calcium phosphate precipitation method of Graham and van der Eb, Virology, 52:456-457 (1978) can be employed. General aspects of mammalian cell host system transfections have been described in U.S. Pat. No. 4,399,216. Transformations into yeast are typically carried out according to the method of Van Solingen et al., J. Bact., 130:946 (1977) and Hsiao et al., Proc. Natl. Acad. Sci. USA, 76:3829 (1979). However, other methods for introducing DNA into cells, such as by nuclear microinjection, electroporation, bacterial protoplast fusion with intact cells, or polycations, e.g., polybrene, polyornithine, may also be used. For various techniques for transforming mammalian cells, see Keown et al., Methods in Enzymology, 185:527-537 (1990) and Mansour et al., Nature, 336:348-352 (1988).

Suitable host cells for cloning or expressing the DNA in the vectors herein include prokaryote, yeast, or higher eukaryote cells. Suitable prokaryotes include but are not limited to eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and K5 772 (ATCC 53,635). Other suitable prokaryotic host cells include Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. These examples are illustrative rather than limiting. Strain W3110 is one particularly preferred host or parent host because it is a common host strain for recombinant DNA product fermentations. Preferably, the host cell secretes minimal amounts of proteolytic enzymes. For example, strain W3110 may be modified to effect a genetic mutation in the genes encoding proteins endogenous to the host, with examples of such hosts including E. coli W3110 strain 1A2, which has the complete genotype tonA; E. coli W3110 strain 9E4, which has the complete genotype tonA ptr3; E. coli W3110 strain 27C7 (ATCC 55,244), which has the complete genotype tonA ptr3 phoA E15 (argF-lac) 169 degP ompT kan^{r}; E. coli W3110 strain 37D6, which has the complete genotype tonA ptr3 phoA E15 (argF-lac)169 degP ompT rbs7 ilvG kan^{r}; E. coli W3110 strain 40B4, which is strain 37D6 with a non-kanamycin resistant degP deletion mutation; and an E. coli strain having mutant periplasmic protease disclosed in U.S. Pat. No. 4,946,783 issued 7 Aug. 1990. Alternatively, in vitro methods of cloning, e.g., PCR or other nucleic acid polymerase reactions, are suitable.

In addition to prokaryotes, eukaryotic microbes such as filamentous fungi or yeast are suitable cloning or expression hosts for DR6 polypeptide-encoding vectors. Saccharomyces cerevisiae is a commonly used lower eukaryotic host microorganism. Others include Schizosaccharomyces pombe (Beach and Nurse (1981) Nature, 290: 140; EP 139,383 published 2 May 1985); Kluyveromyces hosts (U.S. Pat. No. 4,943,529; Fleer et al., Bio/Technology, 9:968-975 (1991)) such as, e.g., K. lactis (MW98-8C, CBS683, CBS4574; Louvencourt et al., J. Bacteriol., 154(2):737-742 (1983)), K. fragilis (ATCC 12,424), K. bulgaricus (ATCC 16,045), K. wickeramii (ATCC 24,178), K. waltii (ATCC 56,500), K. drosophilarum (ATCC 36,906; Van den Berg et al., Bio/Technology, 8:135 (1990)), K. thermotolerans, and K. marxianus; yarrowia (EP 402,226); Pichia pastoris (EP 183,070; Sreekrishna et al., J. Basic Microbiol., 28:265-278 (1988)); Candida; Trichoderma reesia (EP 244,234); Neurospora crassa (Case et al., Proc. Natl. Acad. Sci. USA, 76:5259-5263 (1979)); Schwanniomyces such as Schwanniomyces occidentalis (EP 394,538 published 31 October 1990); and filamentous fungi such as, e.g., Neurospora, Penicillium, Tolypocladium (WO 91/00357 published 10 Jan. 1991), and Aspergillus hosts such as A. nidulans (Ballance et al. (1983) Biochem. Biophys. Res. Commun., 112:284-289; Tilburn et al. (1983) Gene, 26:205-221; Yelton et al. (1984) Proc. Natl. Acad. Sci. USA, 81:1470-1474) and A. niger (Kelly and Hynes (1985) EMBO J. 4:475-479. Methylotropic yeasts are suitable herein and include, but are not limited to, yeast capable of growth on methanol selected from the genera consisting of Hansenula, Candida, Kloeckera, Pichia, Saccharomyces, Torulopsis, and Rhodotorula. A list of specific species that are exemplary of this class of yeasts may be found in C. Anthony, The Biochemistry of Methylotrophs, 269 (1982).

Suitable host cells for the expression of glycosylated DR6, p75 and/or APP polypeptide are derived from multicellular organisms. Examples of invertebrate cells include insect cells such as Drosophila S2 and Spodoptera Sf9, as well as plant cells, such as cell cultures of cotton, corn, potato, soybean, petunia, tomato, and tobacco. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/−DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al. (1982) Annals N.Y. Acad. Sci. 383:44-68); MRC 5 cells; FS4 cells; and a human hepatoma line (Hep G2).

Host cells are transformed with the above-described expression or cloning vectors for DR6 and/or APP polypeptide production and cultured in conventional nutrient media modified as appropriate for inducing promoters, selecting transformants, or amplifying the genes encoding the desired sequences.

### Selection and Use of a Replicable Vector

The nucleic acid (e.g., cDNA or genomic DNA) encoding DR6, p75 and/or APP polypeptide may be inserted into a replicable vector for cloning (amplification of the DNA) or for expression. Various vectors are publicly available. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

The DR6, p75 and/or APP may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the DR6, p75 and/or APP polypeptide-encoding DNA that is inserted into the vector. The signal sequence may be a prokaryotic signal sequence selected, for example, from the group of the alkaline phosphatase, penicillinase, lpp, or heat-stable enterotoxin II leaders. For yeast secretion the signal sequence may be, e.g., the yeast invertase leader, alpha factor leader (including Saccharomyces and Kluyveromyces α-factor leaders, the latter described in U.S. Pat. No. 5,010,182), or acid phosphatase leader, the C. albicans glucoamylase leader (EP 362,179 published 4 Apr. 1990), or the signal described in WO 90/13646 published 15 Nov. 1990. In mammalian cell expression, mammalian signal sequences may be used to direct secretion of the protein, such as signal sequences from secreted polypeptides of the same or related species, as well as viral secretory leaders.

Both expression and cloning vectors contain a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria, the 2μ plasmid origin is suitable for yeast, and various viral origins (SV40, polyoma, adenovirus, VSV or BPV) are useful for cloning vectors in mammalian cells.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli. An example of suitable selectable markers for mammalian cells are those that enable the identification of cells competent to take up the DR6, p75 and/or APP polypeptide-encoding nucleic acid, such as DHFR or thymidine kinase. An appropriate host cell when wild-type DHFR is employed is the CHO cell line deficient in DHFR activity, prepared and propagated as described by Urlaub et al., Proc. Natl. Acad. Sci. USA, 77:4216 (1980). A suitable selection gene for use in yeast is the trp1 gene present in the yeast plasmid YRp7 (Stinchcomb et al., Nature, 282:39 (1979); Kingsman et al., Gene, 7:141 (1979); Tschemper et al., Gene, 10:157 (1980)). The trp1 gene provides a selection marker for a mutant strain of yeast lacking the ability to grow in tryptophan, for example, ATCC No. 44076 or PEP4-1 (Jones, Genetics, 85:12 (1977)).

Expression and cloning vectors usually contain a promoter operably linked to the DR6, p75 and/or APP polypeptide-encoding nucleic acid sequence to direct mRNA synthesis. Promoters recognized by a variety of potential host cells are well known. Promoters suitable for use with prokaryotic hosts include the β-lactamase and lactose promoter systems (Chang et al. (1978) Nature, 275:615; Goeddel et al. (1979) Nature, 281:544), alkaline phosphatase, a tryptophan (trp) promoter system (Goeddel, Nucl. Acids Res., 8:4057 (1980); EP 36,776), and hybrid promoters such as the tac promoter (deBoer et al., Proc. Natl. Acad. Sci. USA, 80:21-25 (1983)). Promoters for use in bacterial systems also will contain a Shine-Dalgarno (S.D.) sequence operably linked to the DNA encoding DR6, p75 and/or APP polypeptide.

Examples of suitable promoting sequences for use with yeast hosts include the promoters for 3-phosphoglycerate kinase (Hitzeman et al., J. Biol. Chem., 255:2073 (1980)) or other glycolytic enzymes (Hess et al., J. Adv. Enzyme Reg., 7:149 (1968); Holland, Biochemistry, 17:4900 (1978)), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657. DR6, p75 and/or APP polypeptide transcription from vectors in mammalian host cells is controlled, for example, by promoters obtained from the genomes of viruses such as polyoma virus, fowlpox virus (UK 2,211,504 published 5 Jul. 1989), adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription of a DNA encoding the DR6, p75 and/or APP polypeptide by higher eukaryotes may be increased by inserting an enhancer sequence into the vector. Enhancers are cis-acting elements of DNA, usually about from 10 to 300 bp, which act on a promoter to increase its transcription. Many enhancer sequences are now known from mammalian genes (globin, elastase, albumin, alpha-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the DR6, p75 and/or APP polypeptide coding sequence, but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding DR6 polypeptide.

Still other methods, vectors, and host cells suitable for adaptation to the synthesis of DR6, p75 and/or APP polypeptide in recombinant vertebrate cell culture are described in Gething et al., Nature, 293:620-625 (1981); Mantei et al., Nature, 281:40-46 (1979); EP 117,060; and EP 117,058.

### Culturing the Host Cells

The host cells used to produce the DR6, p75 and/or APP polypeptide of this invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

### Detecting Gene Amplification/Expression

Gene amplification and/or expression may be measured in a sample directly, for example, by conventional Southern blotting, Northern blotting to quantitate the transcription of mRNA (Thomas, Proc. Natl. Acad. Sci. USA, 77:5201-5205 (1980)), dot blotting (DNA analysis), or in situ hybridization, using an appropriately labeled probe, based on the sequences provided herein. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected.

Gene expression, alternatively, may be measured by immunological methods, such as immunohistochemical staining of cells or tissue sections and assay of cell culture or body fluids, to quantitate directly the expression of gene product. Antibodies useful for immunohistochemical staining and/or assay of sample fluids may be either monoclonal or polyclonal, and may be prepared in any mammal. Conveniently, the antibodies may be prepared against a native sequence DR6 polypeptide or against a synthetic peptide based on the DR6 sequences provided herein or against exogenous sequence fused to DR6 DNA and encoding a specific antibody epitope.

### Purification of DR6 Polypeptide

Forms of DR6, p75 and/or APP polypeptide may be recovered from culture medium or from host cell lysates. If membrane-bound, it can be released from the membrane using a suitable detergent solution (e.g. Triton-X 100) or by enzymatic cleavage. Cells employed in expression of DR6 polypeptide can be disrupted by various physical or chemical means, such as freeze-thaw cycling, sonication, mechanical disruption, or cell lysing agents.

It may be desired to purify DR6, p75 and/or APP polypeptide from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of the DR6 and/or APP polypeptide. Various methods of protein purification may be employed and such methods are known in the art and described for example in Deutscher, Methods in Enzymology, 182 (1990); Scopes, PROTEIN PURIFICATION: PRINCIPLES AND PRACTICE, Springer-Verlag, New York (1982). The purification step(s) selected will depend, for example, on the nature of the production process used and the particular DR6 polypeptide produced.

Soluble forms of DR6, p75 and/or APP may be employed as DR6 antagonists or p75 antagonists in the methods of the invention. Such soluble forms of DR6, p75 and/or APP may comprise modifications, as described below (such as by fusing to an immunoglobulin, epitope tag or leucine zipper). Immunoadhesin molecules are further contemplated for use in the methods herein. DR6, p75 and/or APP immunoadhesins may comprise various forms of DR6, p75 and/or APP, such as the full length polypeptide as well as soluble, extracellular domain forms of the DR6, p75 and/or APP or a fragment thereof. In particular embodiments, the molecule may comprise a fusion of the DR6 polypeptide with an immunoglobulin or a particular region of an immunoglobulin. For a bivalent form of the immunoadhesin, such a fusion could be to the Fc region of an IgG molecule. The Ig fusions preferably include the substitution of a soluble (transmembrane domain deleted or inactivated) form of the polypeptide in place of at least one variable region within an Ig molecule. In a particularly preferred embodiment, the immunoglobulin fusion includes the hinge, CH2 and CH3, or the hinge, CH1, CH2 and CH3 regions of an IgG1 molecule. For the production of immunoglobulin fusions, see also U.S. Pat. No. 5,428,130 issued Jun. 27, 1995 and Chamow et al., TIB TECH, 14:52-60 (1996).

An optional immunoadhesin design combines the binding domain(s) of the adhesin (e.g. a DR6, p75 and/or APP ectodomain) with the Fc region of an immunoglobulin heavy chain. Ordinarily, when preparing the immunoadhesins of the present invention, nucleic acid encoding the binding domain of the adhesin will be fused C-terminally to nucleic acid encoding the N-terminus of an immunoglobulin constant domain sequence, however N-terminal fusions are also possible.

Typically, in such fusions the encoded chimeric polypeptide will retain at least functionally active hinge, C_{H}2 and C_{H}3 domains of the constant region of an immunoglobulin heavy chain. Fusions are also made to the C-terminus of the Fc portion of a constant domain, or immediately N-terminal to the C_{H}1 of the heavy chain or the corresponding region of the light chain. The precise site at which the fusion is made is not critical; particular sites are well known and may be selected in order to optimize the biological activity, secretion, or binding characteristics of the immunoadhesin.

In a preferred embodiment, the adhesin sequence is fused to the N-terminus of the Fc region of immunoglobulin G₁ (IgG₁). It is possible to fuse the entire heavy chain constant region to the adhesin sequence. However, more preferably, a sequence beginning in the hinge region just upstream of the papain cleavage site which defines IgG Fc chemically (i.e. residue 216, taking the first residue of heavy chain constant region to be 114), or analogous sites of other immunoglobulins is used in the fusion. In a particularly preferred embodiment, the adhesin amino acid sequence is fused to (a) the hinge region and C_{H}2 and C_{H}3 or (b) the C_{H}1, hinge, C_{H}2 and C_{H}3 domains, of an IgG heavy chain.

For bispecific immunoadhesins, the immunoadhesins are assembled as multimers, and particularly as heterodimers or heterotetramers. Generally, these assembled immunoglobulins will have known unit structures. A basic four chain structural unit is the form in which IgG, IgD, and IgE exist. A four chain unit is repeated in the higher molecular weight immunoglobulins; IgM generally exists as a pentamer of four basic units held together by disulfide bonds. IgA globulin, and occasionally IgG globulin, may also exist in multimeric form in serum. In the case of multimer, each of the four units may be the same or different. Various exemplary assembled immunoadhesins within the scope herein are schematically diagrammed below:

(a) AC_{L}-AC_{L};

(b) AC_{H}-(AC_{H}, AC_{L}-AC_{H}, AC_{L}-V_{H}C_{H}, or V_{L}C_{L}-AC_{H});

(c) AC_{L}-AC_{H}-(AC_{L}-AC_{H}, AC_{L}-V_{H}C_{H}, V_{L}C_{L}-AC_{H}, or V_{L}C_{L}-V_{H}C_{H})

(d) AC_{L}-V_{H}C_{H}-(AC_{H}, or AC_{L}-V_{H}C_{H}, or V_{L}C_{L}-AC_{H});

(e) V_{L}C_{L}-AC_{H}-(AC_{L}-V_{H}C_{H}, or V_{L}C_{L}-AC_{H}); and

(f) (A-Y)ₙ-(V_{L}C_{L}-V_{H}C_{H})₂,

wherein each A represents identical or different adhesin amino acid sequences;

V_{L} is an immunoglobulin light chain variable domain;

V_{H} is an immunoglobulin heavy chain variable domain;

C_{L} is an immunoglobulin light chain constant domain;

C_{H} is an immunoglobulin heavy chain constant domain;

n is an integer greater than 1;

Y designates the residue of a covalent cross-linking agent.

In the interests of brevity, the foregoing structures only show key features; they do not indicate joining (J) or other domains of the immunoglobulins, nor are disulfide bonds shown. However, where such domains are required for binding activity, they shall be constructed to be present in the ordinary locations which they occupy in the immunoglobulin molecules.

Alternatively, the adhesin sequences can be inserted between immunoglobulin heavy chain and light chain sequences, such that an immunoglobulin comprising a chimeric heavy chain is obtained. In this embodiment, the adhesin sequences are fused to the 3′ end of an immunoglobulin heavy chain in each arm of an immunoglobulin, either between the hinge and the C_{H}2 domain, or between the C_{H}2 and C_{H}3 domains. Similar constructs have been reported by Hoogenboom et al., Mol. Immunol., 28:1027-1037 (1991).

Although the presence of an immunoglobulin light chain is not required in the immunoadhesins of the present invention, an immunoglobulin light chain might be present either covalently associated to an adhesin-immunoglobulin heavy chain fusion polypeptide, or directly fused to the adhesin. In the former case, DNA encoding an immunoglobulin light chain is typically coexpressed with the DNA encoding the adhesin-immunoglobulin heavy chain fusion protein. Upon secretion, the hybrid heavy chain and the light chain will be covalently associated to provide an immunoglobulin-like structure comprising two disulfide-linked immunoglobulin heavy chain-light chain pairs. Methods suitable for the preparation of such structures are, for example, disclosed in U.S. Pat. No. 4,816,567, issued 28 Mar. 1989.

Immunoadhesins are most conveniently constructed by fusing the cDNA sequence encoding the adhesin portion in-frame to an immunoglobulin cDNA sequence. However, fusion to genomic immunoglobulin fragments can also be used (see, e.g. Aruffo et al., Cell, 61:1303-1313 (1990); and Stamenkovic et al., Cell, 66:1133-1144 (1991)). The latter type of fusion requires the presence of Ig regulatory sequences for expression. cDNAs encoding IgG heavy-chain constant regions can be isolated based on published sequences from cDNA libraries derived from spleen or peripheral blood lymphocytes, by hybridization or by polymerase chain reaction (PCR) techniques. The cDNAs encoding the “adhesin” and the immunoglobulin parts of the immunoadhesin are inserted in tandem into a plasmid vector that directs efficient expression in the chosen host cells.

In another embodiment, the DR6 antagonist may be covalently modified by linking the receptor polypeptide to one of a variety of nonproteinaceous polymers, e.g., polyethylene glycol (PEG), polypropylene glycol, or polyoxyalkylenes, in the manner set forth in U.S. Pat. No. 4,640,835; 4,496,689; 4,301,144; 4,670,417; 4,791,192 or 4,179,337, or other like molecules such as polyglutamate. Such pegylated forms may be prepared using techniques known in the art.

Leucine zipper forms of these molecules are also contemplated by the invention. “Leucine zipper” is a term in the art used to refer to a leucine rich sequence that enhances, promotes, or drives dimerization or trimerization of its fusion partner (e.g., the sequence or molecule to which the leucine zipper is fused or linked to). Various leucine zipper polypeptides have been described in the art. See, e.g., Landschulz et al., Science, 240:1759 (1988); U.S. Pat. No. 5,716,805; WO 94/10308; Hoppe et al., FEBS Letters, 344:1991 (1994); Maniatis et al., Nature, 341:24 (1989). Those skilled in the art will appreciate that a leucine zipper sequence may be fused at either the 5′ or 3′ end of the DR6 or p75 molecule.

The DR6, p75 and/or APP polypeptides of the present invention may also be modified in a way to form chimeric molecules by fusing the polypeptide to another, heterologous polypeptide or amino acid sequence. Preferably, such heterologous polypeptide or amino acid sequence is one which acts to oligimerize the chimeric molecule. In one embodiment, such a chimeric molecule comprises a fusion of the DR6, p75 and/or APP polypeptide with a tag polypeptide which provides an epitope to which an anti-tag antibody can selectively bind. The epitope tag is generally placed at the amino- or carboxyl-terminus of the polypeptide. The presence of such epitope-tagged forms of the polypeptide can be detected using an antibody against the tag polypeptide. Also, provision of the epitope tag enables the polypeptide to be readily purified by affinity purification using an anti-tag antibody or another type of affinity matrix that binds to the epitope tag. Various tag polypeptides and their respective antibodies are well known in the art. Examples include poly-histidine (poly-his) or poly-histidine-glycine (poly-his-gly) tags; the flu HA tag polypeptide and its antibody 12CA5 (Field et al., Mol. Cell. Biol., 8:2159-2165 (1988)); the c-myc tag and the 8F9, 3C7, 6E10, G4, B7 and 9E10 antibodies thereto (Evan et al., Mol. Cell. Biol., 5:3610-3616 (1985)); and the Herpes Simplex virus glycoprotein D (gD) tag and its antibody (Paborsky et al., Protein Engineering, 3(6):547-553 (1990)). Other tag polypeptides include the Flag-peptide (Hopp et al., Bio Technology, 6:1204-1210 (1988)); the KT3 epitope peptide (Martin et al., Science, 255:192-194 (1992)); an alpha-tubulin epitope peptide (Skinner et al., J. Biol. Chem., 266:15163-15166 (1991)); and the T7 gene 10 protein peptide tag (Lutz-Freyermuth et al., Proc. Natl. Acad. Sci. USA, 87:6393-6397 (1990)).

### Anti-DR6, Anti-p75 and Anti-APP Antibodies

In other embodiments of the invention, DR6, p75 and/or APP antibodies are provided. Exemplary antibodies include polyclonal, monoclonal, humanized, bispecific, and heteroconjugate antibodies. In some embodiments, these anti-DR6, p75 and/or APP antibodies are preferably DR6 antagonist antibodies.

### Polyclonal Antibodies

The antibodies of the invention may comprise polyclonal antibodies. Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include DR6, p75 and/or APP polypeptide (e.g. a DR6, p75 and/or APP ECD) or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation. The mammal can then be bled, and the serum assayed for DR6 and/or APP antibody titer. If desired, the mammal can be boosted until the antibody titer increases or plateaus.

### Monoclonal Antibodies

The antibodies of the invention may, alternatively, be monoclonal antibodies. Monoclonal antibodies may be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The immunizing agent will typically include the DR6, p75 and/or APP polypeptide (e.g. a DR6, p75 and/or APP ECD) or a fusion protein thereof, such as a DR6 ECD-IgG, p75 ECD-IgG and/or APP sAPP-IgG fusion protein.

Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, MONOCLONAL ANTIBODIES: PRINCIPLES AND PRACTICE, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. An example of such a murine myeloma cell line is P3X63Ag8U.1, (ATCC CRL 1580). Human myeloma and mouse-human heteromyeloma cell lines also have been described for the production of human monoclonal antibodies (Kozbor, J. Immunol., 133:3001 (1984); Brodeur et al., MONOCLONAL ANTIBODY PRODUCTION TECHNIQUES AND APPLICATIONS, Marcel Dekker, Inc., New York, (1987) pp. 51-63).

The culture medium in which the hybridoma cells are cultured can then be assayed for the presence of monoclonal antibodies directed against DR6, p75 and/or APP. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980) or by way of BiaCore analysis.

After the desired hybridoma cells are identified, the clones may be subcloned by limiting dilution procedures and grown by standard methods (Goding, supra). Suitable culture media for this purpose include, for example, Dulbecco's Modified Eagle's Medium or RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences, Morrison, et al., Proc. Nat. Acad. Sci. USA 81, 6851 (1984), or by covalently joining to the immunoglobulin coding sequence all or part of the coding sequence for a non-immunoglobulin polypeptide. In that manner, “chimeric” or “hybrid” antibodies are prepared that have the binding specificity of an anti-DR6 monoclonal antibody herein.

Typically such non-immunoglobulin polypeptides are substituted for the constant domains of an antibody of the invention, or they are substituted for the variable domains of one antigen-combining site of an antibody of the invention to create a chimeric bivalent antibody comprising one antigen-combining site having specificity for DR6 and another antigen-combining site having specificity for a different antigen.

Chimeric or hybrid antibodies also may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate.

Single chain Fv fragments may also be produced, such as described in Iliades et al., FEBS Letters, 409:437-441 (1997). Coupling of such single chain fragments using various linkers is described in Kortt et al., Protein Engineering, 10:423-433 (1997). A variety of techniques for the recombinant production and manipulation of antibodies are well known in the art. Illustrative examples of such techniques that are typically utilized by skilled artisans are described in greater detail below.

### Humanized Antibodies

Generally, a humanized antibody has one or more amino acid residues introduced into it from a non-human source. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody.

Accordingly, such “humanized” antibodies are chimeric antibodies wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

It is important that antibodies be humanized with retention of high affinity for the antigen and other favorable biological properties. To achieve this goal, according to a preferred method, humanized antibodies are prepared by a process of analysis of the parental sequences and various conceptual humanized products using three dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e. the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the CDR residues are directly and most substantially involved in influencing antigen binding.

### Human Antibodies

Human monoclonal antibodies can be made by the hybridoma method. Human myeloma and mouse-human heteromyeloma cell lines for the production of human monoclonal antibodies have been described, for example, by Kozbor, J. Immunol. 133, 3001 (1984), and Brodeur, et al., MONOCLONAL ANTIBODY PRODUCTION TECHNIQUES AND APPLICATIONS, pp. 51-63 (Marcel Dekker, Inc., New York, 1987).

It is now possible to produce transgenic animals (e.g. mice) that are capable, upon immunization, of producing a repertoire of human antibodies in the absence of endogenous immunoglobulin production. For example, it has been described that the homozygous deletion of the antibody heavy chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g. Jakobovits et al., Proc. Natl. Acad. Sci. USA 90, 2551-255 (1993); Jakobovits et al., Nature 362, 255-258 (1993).

Mendez et al. (Nature Genetics 15: 146-156 (1997)) have further improved the technology and have generated a line of transgenic mice designated as “Xenomouse II” that, when challenged with an antigen, generates high affinity fully human antibodies. This was achieved by germ-line integration of megabase human heavy chain and light chain loci into mice with deletion into endogenous J_{H} segment as described above. The Xenomouse II harbors 1,020 kb of human heavy chain locus containing approximately 66 V_{H} genes, complete D_{H} and J_{H} regions and three different constant regions (μ, δ and χ), and also harbors 800 kb of human κ locus containing 32 Vκ genes, Jκ segments and Cκ genes. The antibodies produced in these mice closely resemble that seen in humans in all respects, including gene rearrangement, assembly, and repertoire. The human antibodies are preferentially expressed over endogenous antibodies due to deletion in endogenous J_{H} segment that prevents gene rearrangement in the murine locus.

Alternatively, the phage display technology (McCafferty et al., Nature 348, 552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats; for their review see, e.g. Johnson, Kevin S. and Chiswell, David J., Current Opinion in Structural Biology 3, 564-571 (1993). Several sources of V-gene segments can be used for phage display. Clackson et al., Nature 352, 624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222, 581-597 (1991), or Griffith et al., EMBO J. 12, 725-734 (1993). In a natural immune response, antibody genes accumulate mutations at a high rate (somatic hypermutation). Some of the changes introduced will confer higher affinity, and B cells displaying high-affinity surface immunoglobulin are preferentially replicated and differentiated during subsequent antigen challenge. This natural process can be mimicked by employing the technique known as “chain shuffling” (Marks et al., Bio/Technol. 10, 779-783 [1992]). In this method, the affinity of “primary” human antibodies obtained by phage display can be improved by sequentially replacing the heavy and light chain V region genes with repertoires of naturally occurring variants (repertoires) of V domain genes obtained from unimmunized donors. This technique allows the production of antibodies and antibody fragments with affinities in the nM range. A strategy for making very large phage antibody repertoires (also known as “the mother-of-all libraries”) has been described by Waterhouse et al., Nucl. Acids Res. 21, 2265-2266 (1993). Gene shuffling can also be used to derive human antibodies from rodent antibodies, where the human antibody has similar affinities and specificities to the starting rodent antibody. According to this method, which is also referred to as “epitope imprinting”, the heavy or light chain V domain gene of rodent antibodies obtained by phage display technique is replaced with a repertoire of human V domain genes, creating rodent-human chimeras. Selection on antigen results in isolation of human variable capable of restoring a functional antigen-binding site, i.e. the epitope governs (imprints) the choice of partner. When the process is repeated in order to replace the remaining rodent V domain, a human antibody is obtained (see PCT patent application WO 93/06213, published 1 Apr. 1993). Unlike traditional humanization of rodent antibodies by CDR grafting, this technique provides completely human antibodies, which have no framework or CDR residues of rodent origin.

As discussed in detail below, the antibodies of the invention may optionally comprise monomeric, antibodies, dimeric antibodies, as well as multivalent forms of antibodies. Those skilled in the art may construct such dimers or multivalent forms by techniques known in the art and using the DR6 and/or APP antibodies herein. Methods for preparing monovalent antibodies are also well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking

### Bispecific Antibodies

Bispecific antibodies are monoclonal, preferably human or humanized, antibodies that have binding specificities for at least two different antigens. In the present case, one of the binding specificities is for the DR6 receptor, the other one is for any other antigen, and preferably for another receptor or receptor subunit. In one embodiment, the other antigen is p75. Methods for making bispecific antibodies are known in the art. Traditionally, the recombinant production of bispecific antibodies is based on the coexpression of two immunoglobulin heavy chain-light chain pairs, where the two heavy chains have different specificities (Millstein and Cuello, Nature 305, 537-539 (1983)). Because of the random assortment of immunoglobulin heavy and light chains, these hybridomas (quadromas) produce a potential mixture of 10 different antibody molecules, of which only one has the correct bispecific structure. The purification of the correct molecule, which is usually done by affinity chromatography steps, is rather cumbersome, and the product yields are low. Similar procedures are disclosed in PCT application publication No. WO 93/08829 (published 13 May 1993), and in Traunecker et al., EMBO J. 10, 3655-3659 (1991).

According to a different and more preferred approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2 and CH3 regions. It is preferred to have the first heavy chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are cotransfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance. In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in PCT Publication No. WO 94/04690, published on Mar. 3, 1994.

For further details of generating bispecific antibodies see, for example, Suresh et al., Meth. Enzymol. 121, 210 (1986).

### Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (PCT application publication Nos. WO 91/00360 and WO 92/200373; EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

### Antibody Fragments

In certain embodiments, the anti-DR6, anti-p75 and/or anti-APP antibody (including murine, human and humanized antibodies, and antibody variants) is an antibody fragment. Various techniques have been developed for the production of antibody fragments. Traditionally, these fragments were derived via proteolytic digestion of intact antibodies (see, e.g., Morimoto et al., J. Biochem. Biophys. Methods 24:107-117 (1992) and Brennan et al., Science 229:81 (1985)). However, these fragments can now be produced directly by recombinant host cells. For example, Fab′-SH fragments can be directly recovered from E. coli and chemically coupled to form F(ab′)₂ fragments (Carter et al., Bio/Technology 10:163-167 (1992)). In another embodiment, the F(ab′)₂ is formed using the leucine zipper GCN4 to promote assembly of the F(ab′)₂ molecule. According to another approach, Fv, Fab or F(ab′)₂ fragments can be isolated directly from recombinant host cell culture. A variety of techniques for the production of antibody fragments will be apparent to the skilled practitioner. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields an F(ab′)₂ fragment that has two antigen combining sites and is still capable of cross-linking antigen.

The Fab fragments produced in the antibody digestion also contain the constant domains of the light chain and the first constant domain (CH₁) of the heavy chain. Fab′ fragments differ from Fab fragments by the addition of a few residues at the carboxy terminus of the heavy chain CHᵢ domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

### Glycosylation Variants of Antibodies

Antibodies are glycosylated at conserved positions in their constant regions (Jefferis and Lund, Chem. Immunol. 65:111-128 (1997); Wright and Morrison, TibTECH 15:26-32 [1997]). The oligosaccharide side chains of the immunoglobulins affect the protein's function (Boyd et al., Mol. Immunol. 32:1311-1318 (1996); Wittwe and Howard, Biochem. 29:4175-4180 (1990)), and the intramolecular interaction between portions of the glycoprotein which can affect the conformation and presented three-dimensional surface of the glycoprotein (Hefferis and Lund, supra; Wyss and Wagner, Current Opin. Biotech. 7:409-416 (1996)). Oligosaccharides may also serve to target a given glycoprotein to certain molecules based upon specific recognition structures. For example, it has been reported that in agalactosylated IgG, the oligosaccharide moiety ‘flips’ out of the inter-CH2 space and terminal N-acetylglucosamine residues become available to bind mannose binding protein (Malhotra et al., Nature Med. 1:237-243 (1995)). Removal by glycopeptidase of the oligosaccharides from CAMPATH-1H (a recombinant humanized murine monoclonal IgG1 antibody which recognizes the CDw52 antigen of human lymphocytes) produced in Chinese Hamster Ovary (CHO) cells resulted in a complete reduction in complement mediated lysis (CMCL) (Boyd et al., Mol. Immunol. 32:1311-1318 [1996]), while selective removal of sialic acid residues using neuraminidase resulted in no loss of DMCL. Glycosylation of antibodies has also been reported to affect antibody-dependent cellular cytotoxicity (ADCC). In particular, CHO cells with tetracycline-regulated expression of β(1,4)-N-acetylglucosaminyltransferase III (GnTIII), a glycosyltransferase catalyzing formation of bisecting GlcNAc, was reported to have improved ADCC activity (Umana et al., Mature Biotech. 17:176-180 (1999)).

Glycosylation variants of antibodies are variants in which the glycosylation pattern of an antibody is altered. By altering is meant deleting one or more carbohydrate moieties found in the antibody, adding one or more carbohydrate moieties to the antibody, changing the composition of glycosylation (glycosylation pattern), the extent of glycosylation, etc. Glycosylation variants may, for example, be prepared by removing, changing and/or adding one or more glycosylation sites in the nucleic acid sequence encoding the antibody.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

The glycosylation (including glycosylation pattern) of antibodies may also be altered without altering the underlying nucleotide sequence. Glycosylation largely depends on the host cell used to express the antibody. Since the cell type used for expression of recombinant glycoproteins, e.g. antibodies, as potential therapeutics is rarely the native cell, significant variations in the glycosylation pattern of the antibodies can be expected (see, e.g. Hse et al., J. Biol. Chem. 272:9062-9070 (1997)). In addition to the choice of host cells, factors which affect glycosylation during recombinant production of antibodies include growth mode, media formulation, culture density, oxygenation, pH, purification schemes and the like. Various methods have been proposed to alter the glycosylation pattern achieved in a particular host organism including introducing or overexpressing certain enzymes involved in oligosaccharide production (U.S. Pat. Nos. 5,047,335; 5,510,261 and 5,278,299). Glycosylation, or certain types of glycosylation, can be enzymatically removed from the glycoprotein, for example using endoglycosidase H (Endo H). In addition, the recombinant host cell can be genetically engineered, e.g. make defective in processing certain types of polysaccharides. These and similar techniques are well known in the art.

The glycosylation structure of antibodies can be readily analyzed by conventional techniques of carbohydrate analysis, including lectin chromatography, NMR, Mass spectrometry, HPLC, GPC, monosaccharide compositional analysis, sequential enzymatic digestion, and HPAEC-PAD, which uses high pH anion exchange chromatography to separate oligosaccharides based on charge. Methods for releasing oligosaccharides for analytical purposes are also known, and include, without limitation, enzymatic treatment (commonly performed using peptide-N-glycosidase F/endo-β-galactosidase), elimination using harsh alkaline environment to release mainly O-linked structures, and chemical methods using anhydrous hydrazine to release both N- and O-linked oligosaccharides.

### Exemplary Antibodies

As described in the Examples below, anti-DR6 monoclonal antibodies have been identified. In optional embodiments, the DR6 antibodies of the invention will have the same biological characteristics as any of the anti-DR6, anti-p75 and/or anti-APP antibodies specifically disclosed herein.

The term “biological characteristics” is used to refer to the in vitro and/or in vivo activities or properties of the monoclonal antibody, such as the ability to specifically bind to DR6 or to block, inhibit, or neutralize DR6 activation. The properties and activities of the DR6, p75 and/or APP antibodies are further described in the Examples below. Optionally, the monoclonal antibodies of the present invention will have the same biological characteristics as any of the antibodies specifically characterized in the Examples below, and/or bind to the same epitope(s) as these antibodies. This can be determined by conducting various assays, such as described herein and in the Examples. For instance, to determine whether a monoclonal antibody has the same specificity as the DR6, p75 and/or APP antibodies specifically referred to herein, one can compare its activity in competitive binding assays. In addition, an epitope to which a particular anti-DR6, p75 and/or APP antibody binds can be determined by crystallography study of the complex between DR6, p75 and/or APP and the antibody in question.

The DR6, p75 and/or APP antibodies, as described herein, will preferably possess the desired DR6, p75 or APP antagonistic activity. Such antibodies may include but are not limited to chimeric, humanized, human, and affinity matured antibodies. As described above, the DR6, p75 and/or APP antibodies may be constructed or engineered using various techniques to achieve these desired activities or properties.

Additional embodiments of the invention include an anti-DR6 receptor, anti-p75 and/or anti-APP ligand antibody disclosed herein which is linked to one or more non-proteinaceous polymers selected from the group consisting of polyethylene glycol, polypropylene glycol, and polyoxyalkylene. Optionally, an anti-DR6 receptor, anti-p75 and/or anti-APP ligand antibody disclosed herein is glycosylated or alternatively, unglycosylated.

The antibodies of the invention include “cross-linked” DR6, p75 and/or APP antibodies. The term “cross-linked” as used herein refers to binding of at least two IgG molecules together to form one (or single) molecule. The DR6, p75 and/or APP antibodies may be cross-linked using various linker molecules, preferably the DR6, p75 and/or APP antibodies are cross-linked using an anti-IgG molecule, complement, chemical modification or molecular engineering. It is appreciated by those skilled in the art that complement has a relatively high affinity to antibody molecules once the antibodies bind to cell surface membrane. Accordingly, for example, it is believed that complement may be used as a cross-linking molecule to link two or more anti-DR6 antibodies bound to cell surface membrane.

The invention also provides isolated nucleic acids encoding DR6, p75 and/or APP antibodies as disclosed herein, vectors and host cells comprising the nucleic acid, and recombinant techniques for the production of the antibody.

For recombinant production of the antibody, the nucleic acid encoding it is isolated and inserted into a replicable vector for further cloning (amplification of the DNA) or for expression. DNA encoding the antibody is readily isolated and sequenced using conventional procedures (e.g., by using oligonucleotide probes that are capable of binding specifically to genes encoding the antibody). Many vectors are available. The vector components generally include, but are not limited to, one or more of the following: a signal sequence, an origin of replication, one or more marker genes, an enhancer element, a promoter, and a transcription termination sequence.

The methods herein include methods for the production of chimeric or recombinant anti-DR6 and/or APP antibodies which comprise the steps of providing a vector comprising a DNA sequence encoding an anti-DR6, anti-p75 and/or anti-APP antibody light chain or heavy chain (or both a light chain and a heavy chain), transfecting or transforming a host cell with the vector, and culturing the host cell(s) under conditions sufficient to produce the recombinant anti-DR6 antibody, anti-p75 antibody and/or anti-APP antibody product.

### Formulations of DR6 Antagonists

In the preparation of typical formulations herein, it is noted that the recommended quality or “grade” of the components employed will depend on the ultimate use of the formulation. For therapeutic uses, it is preferred that the component(s) are of an allowable grade (such as “GRAS”) as an additive to pharmaceutical products.

In certain embodiments, there are provided compositions comprising DR6 and, optionally, the p75 antagonist(s) and one or more excipients which provide sufficient ionic strength to enhance solubility and/or stability of the DR6 antagonist, wherein the composition has a pH of 6 (or about 6) to 9 (or about 9). The DR6 and p75 antagonists may be prepared by any suitable method to achieve the desired purity of the protein, for example, according to the above methods. In certain embodiments, the antagonist is recombinantly expressed in host cells or prepared by chemical synthesis. The concentration of the DR6 or p75 antagonist in the formulation may vary depending, for instance, on the intended use of the formulation. Those skilled in the art can determine without undue experimentation the desired concentration of the DR6 or p75 antagonist.

The one or more excipients in the formulations which provide sufficient ionic strength to enhance solubility and/or stability of the DR6 or p75 antagonist is optionally a polyionic organic or inorganic acid, aspartate, sodium sulfate, sodium succinate, sodium acetate, sodium chloride, Captisol™, Tris, arginine salt or other amino acids, sugars and polyols such as trehalose and sucrose. Preferably the one or more excipients in the formulations which provide sufficient ionic strength is a salt. Salts which may be employed include but are not limited to sodium salts and arginine salts. The type of salt employed and the concentration of the salt are preferably such that the formulation has a relatively high ionic strength which allows the DR6 antagonist in the formulation to be stable. Optionally, the salt is present in the formulation at a concentration of about 20 mM to about 0.5 M.

The composition preferably has a pH of 6 (or about 6) to 9 (or about 9), more preferably about 6.5 to about 8.5, and even more preferably about 7 to about 7.5. In a preferred aspect of this embodiment, the composition will further comprise a buffer to maintain the pH of the composition at least about 6 to about 8. Examples of buffers which may be employed include but are not limited to Tris, HEPES, and histidine. When employing Tris, the pH may optionally be adjusted to about 7 to 8.5. When employing Hepes or histidine, the pH may optionally be adjusted to about 6.5 to 7. Optionally, the buffer is employed at a concentration of about 5 mM to about 50 mM in the formulation. Particularly for liquid formulations (or reconstituted lyophilized formulations), it may be desirable to include one or more surfactants in the composition. Such surfactants may, for instance, comprise a non-ionic surfactant like TWEEN™ or PLURONICS™ (e.g., polysorbate or poloxamer). Preferably, the surfactant comprises polysorbate 20 (“Tween 20”). The surfactant will optionally be employed at a concentration of about 0.005% to about 0.2%.

The formulations of the present invention may include, in addition to DR6 antagonist(s) and those components described above, further various other excipients or components. Optionally, the formulation may contain, for parenteral administration, a pharmaceutically or parenterally acceptable carrier, i.e., one that is non-toxic to recipients at the dosages and concentrations employed and is compatible with other ingredients of the formulation. Optionally, the carrier is a parenteral carrier, such as a solution that is isotonic with the blood of the recipient. Examples of such carrier vehicles include water, saline or a buffered solution such as phosphate-buffered saline (PBS), Ringer's solution, and dextrose solution. Various optional pharmaceutically acceptable carriers, excipients, or stabilizers are described further in Remington's Pharmaceutical Sciences, 16th edition, Osol, A. ed. (1980).

The formulations herein also may contain one or more preservatives. Examples include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols, alkyl parabens such as methyl or propyl paraben, and m-cresol. Antioxidants include ascorbic acid and methionine; preservatives (such as octadecyldimethylbenzyl ammonium chloride; hexamethonium chloride; benzalkonium chloride, benzethonium chloride; butyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; sugars such as sucrose, mannitol, trehalose or sorbitol; or polyethylene glycol (PEG)

The compositions of the invention may comprise liquid formulations (liquid solutions or liquid suspensions), and lyophilized formulations, as well as suspension formulations.

The final formulation, if a liquid, is preferably stored frozen at <20° C. Alternatively, the formulation can be lyophilized and provided as a powder for reconstitution with water for injection that optionally may be stored at 2-30° C.

The formulation to be used for therapeutic administration must be sterile. Sterility is readily accomplished by filtration through sterile filtration membranes (e.g., 0.2 micron membranes). Therapeutic compositions generally are placed into a container having a sterile access port, for example, an intravenous solution bag or vial having a stopper pierceable by a hypodermic injection needle.

The composition ordinarily will be stored in single unit or multi-dose containers, for example, sealed ampules or vials, as an aqueous solution or as a lyophilized formulation for reconstitution. The containers may any available containers in the art and filled using conventional methods. Optionally, the formulation may be included in an injection pen device (or a cartridge which fits into a pen device), such as those available in the art (see, e.g., U.S. Pat. No. 5,370,629), which are suitable for therapeutic delivery of the formulation. An injection solution can be prepared by reconstituting the lyophilized DR6 antagonist formulation using, for example, Water-for-Injection.

### Therapies Using DR6 Antagonist(s)

The DR6 antagonists of the invention have various utilities. DR6 antagonists are useful in the diagnosis and treatment of psychiatric disorders. Diagnosis in mammals of the various pathological conditions described herein can be made by the skilled practitioner. Diagnostic techniques are available in the art which allow, e.g., for the diagnosis or detection of various psychiatric disorders in a mammal.

Psychiatric disorders contemplated for treatment by the present invention include addiction and schizophrenia. Cognitive disorders contemplated for treatment by the present invention include Tourette Syndrome, Lett Syndrome, Fragile-X Syndrome, and autism. It is contemplated that the compositions and methods of the invention could be employed to treat normal, aging patients to maintain and perhaps improve cognition during the aging process.

In the methods of the invention, the DR6 antagonist is preferably administered to the mammal in a carrier; preferably a pharmaceutically-acceptable carrier. Suitable carriers and their formulations are described in REMINGTON'S PHARMACEUTICAL SCIENCES, 16th ed., 1980, Mack Publishing Co., edited by Osol et al. Typically, an appropriate amount of a pharmaceutically-acceptable salt is used in the formulation to render the formulation isotonic. Examples of the carrier include saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of DR6 antagonist being administered.

The DR6 antagonist can be administered to the mammal by injection (e.g., intravenous, intraperitoneal, subcutaneous, intramuscular, intraportal), orally, or by other methods such as infusion that ensure its delivery to the bloodstream in an effective form. The DR6 antagonist may also be administered by isolated perfusion techniques, such as isolated tissue perfusion, or by intrathecal, intraoccularly, or lumbar puncture to exert local therapeutic effects. DR6 antagonists that do not readily cross the blood-brain barrier may be given directly, e.g., intracerebrally or into the spinal cord space or otherwise, that will transport them across the barrier. Effective dosages and schedules for administering the DR6 antagonist may be determined empirically, and making such determinations is within the skill in the art. Those skilled in the art will understand that the dosage of DR6 antagonist that must be administered will vary depending on, for example, the mammal which will receive the antagonist, the route of administration, the particular type of antagonist used and other drugs being administered to the mammal. Guidance in selecting appropriate doses is found in the literature, for example, on therapeutic uses of antibodies, e.g., HANDBOOK OF MONOCLONAL ANTIBODIES, Ferrone et al., eds., Noges Publications, Park Ridge, N.J., (1985) ch. 22 and pp. 303-357; Smith et al., ANTIBODIES IN HUMAN DIAGNOSIS AND THERAPY, Haber et al., eds., Raven Press, New York (1977) pp. 365-389. A typical daily dosage of DR6 antibody used alone might range from about 1 μg/kg to up to 100 mg/kg of body weight or more per day, depending on the factors mentioned above.

The DR6 antagonist may also be administered to the mammal in combination with one or more other therapeutic agents. It appears that APP also binds to a lesser extent to p75 (EC₅₀=˜300 nM by ELISA). Thus, it may be advantageous to treat psychiatric and cognitive disorders with a combination of DR6 antagonists as well as with p75 antagonists. Other therapeutic agents may be further combined with DR6 antagonists, optionally in combination with p75 antagonists. Examples of such other therapeutic agents include epidermal growth factor receptor (EGFR) inhibitors, e.g., compounds that bind to or otherwise interact directly with EGFR and prevent or reduce its signalling activity, such as Tarceva, antibodies like C225, also referred to as cetuximab and Erbitux® (ImClone Systems Inc.), fully human ABX-EGF (panitumumab, Abgenix Inc.), as well as fully human antibodies known as E1.1, E2.4, E2.5, E6.2, E6.4, E2.11, E6. 3 and E7.6. 3 and described in U.S. Pat. No. 6,235,883; MDX-447 (Medarex Inc), as well as EGFR small molecule inhibitors such as compounds described in U.S. Pat. No. 5,616,582, U.S. Pat. No. 5,457,105, U.S. Pat. No. 5,475,001, U.S. Pat. No. 5,654,307, U.S. Pat. No. 5,679,683, U.S. Pat. No. 6,084,095, U.S. Pat. No. 6,265,410, U.S. Pat. No. 6,455,534, U.S. Pat. No. 6,521,620, U.S. Pat. No. 6,596,726, U.S. Pat. No. 6,713,484, U.S. Pat. No. 5,770,599, U.S. Pat. No. 6,140,332, U.S. Pat. No. 5,866,572, U.S. Pat. No. 6,399,602, U.S. Pat. No. 6,344,459, U.S. Pat. No. 6,602,863, U.S. Pat. No. 6,391,874, WO9814451, WO9850038, WO9909016, WO9924037, U.S. Pat. No. 6,344,455, U.S. Pat. No. 5,760,041, U.S. Pat. No. 6,002,008, U.S. Pat. No. 5,747,498; particular small molecule EGFR inhibitors include OSI-774 (CP-358774, erlotinib, OSI Pharmaceuticals); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); Iressa (ZD1839, gefitinib, 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); and EKB-569 (N-[4-[(3-chloro-4-fluorophenyl)amino]-3-cyano-7-ethoxy-6-quinolinyl]-4-(dimethylamino)-2-butenamide). Other therapeutic agents that may be employed include apoptosis inhibitors, particularly intracellular apoptosis inhibitors, e.g. caspase inhibitors such as caspase-3, caspase-6, or caspase-8 inhibitors, Bid inhibitors, Bax inhibitors or any combination thereof. Examples of suitable inhibitors are caspase inhibitors in general, dipeptide inhibitors, carbamate inhibitors, substituted aspartic acid acetals, heterocyclyldicarbamides, quinoline-(di-, tri-, tetrapeptide) derivatives, substituted 2-aminobenzamide caspase inhibitors, substituted a-hydroxy acid caspase inhibitors, inhibition by nitrosylation; CASP-1; CASP-3: protein-inhibitors, antisense molecules, nicotinyl-aspartyl-ketones, y-ketoacid dipeptide derivatives, CASP-8: antisense molecules, interacting proteins CASP-9, CASP2: antisense molecules; CASP-6: antisense molecules; CASP-7: antisense molecules; and CASP-12 inhibitors. Further examples are mitochondrial inhibitors such as Bcl-2-modulating factor; Bcl-2 mutant peptides derived from Bad, Bad, BH3-interacting domain death agonist, Bax inhibitor proteins and BLK genes and gene products. Further suitable intracellular modulators of apoptosis are modulators of CASP9/Apaf-1 association, antisense modulators of Apaf-1 expression, peptides for inhibition of apoptosis, anti-apoptotic compositions comprising the R1 subunit of Herpes Simplex virus, MEKK1 and fragments thereof, modulators of Survivin, modulators of inhibitors of apoptosis and HIAP2. Further examples of such agents include Minocycline (Neuroapoptosis Laboratory which inhibits cytochrome c release from mitochondria and blocks caspase-3 mRNA upregulation, Pifithrin alpha (UIC) which is a p53 inhibitor, CEP-1346 (Cephalon Inc.) which is a JNK pathway inhibitor, TCH346 (Novartis) which inhibits pro-apoptotic GAPDH signaling, IDN6556 (Idun Pharmaceuticals) which is a pan-caspase inhibitor; AZQs (AstraZeneca) which is a caspase-3 inhibitor, HMR-3480 (Aventis Pharma) which is a caspase-1/-4 inhibitor, and Activase/TPA (Genentech) which dissolves blood clots (thrombolytic drug).

Further suitable agents which may be administered, in addition to DR6 antagonist, include BACE inhibitors, cholinesterase inhibitors (such as Donepezil, Galantamine, Rivastigmine, Tacrine), NMDA receptor antagonists (such as Memantine), Aβ aggregation inhibitors, antioxidants, γ-secretase modulators, NGF mimics or NGF gene therapy, PPARγ agonists, HMG-CoA reductase inhibitors (statins), ampakines, calcium channel blockers, GABA receptor antagonists, glycogen synthase kinase inhibitors, intravenous immunoglobulin, muscarinic receptor agonists, nicotinic receptor modulators, active or passive Aβ immunization, phosphodiesterase inhibitors, serotonin receptor antagonists and anti-Aβ antibodies (see, eg., WO 2007/062852; WO 2007/064972; WO 2003/040183; WO 1999/06066; WO 2006/081171; WO 1993/21526; EP 0276723B1; WO 2005/028511; WO 2005/082939).

The DR6 antagonist may be administered sequentially or concurrently with the one or more other therapeutic agents. The amounts of DR6 antagonist and therapeutic agent depend, for example, on what type of drugs are used, the pathological condition being treated, and the scheduling and routes of administration but would generally be less than if each were used individually.

Following administration of DR6 antagonist and, optionally, the p75 antagonist to the mammal, the mammal's physiological condition can be monitored in various ways well known to the skilled practitioner.

The therapeutic effects of the DR6 antagonists and, optionally, the p75 antagonist of the invention can be examined in in vitro assays and using in vivo animal models.

### Kits and Articles of Manufacture

In further embodiments of the invention, there are provided articles of manufacture and kits containing materials useful for treating psychiatric disorders and cognitive disorders. The article of manufacture comprises a container with a label. Suitable containers include, for example, bottles, vials, and test tubes. The containers may be formed from a variety of materials such as glass or plastic, and are preferably sterilized. The container holds a composition having an active agent which is effective for treating psychiatric and cognitive disorders. The active agent in the composition is a DR6 antagonist and preferably, comprises anti-DR6 monoclonal antibodies or anti-APP monoclonal antibodies. In some embodiments, another active agent in the composition is a p75 antagonist and preferably, comprises anti-p75 monoclonal antibodies or anti-APP monoclonal antibodies. The label on the container indicates that the composition is used for treating psychiatric and cognitive disorders, and may also indicate directions for either in vivo or in vitro use, such as those described above. The article of manufacture or kit optionally further includes a package insert, which refers to instructions customarily included in commercial packages of therapeutic products, that contain information about the indications, usage, dosage, administration, contraindications, other therapeutic products to be combined with the packaged product, and/or warnings concerning the use of such therapeutic products, etc.

The kit of the invention comprises the container described above and a second container comprising a buffer. It may further include other materials desirable from a commercial and user standpoint, including other buffers, diluents, filters, needles, and syringes.

### EXAMPLES

Various aspects of the invention are further described and illustrated by way of the examples that follow, none of which are intended to limit the scope of the invention.

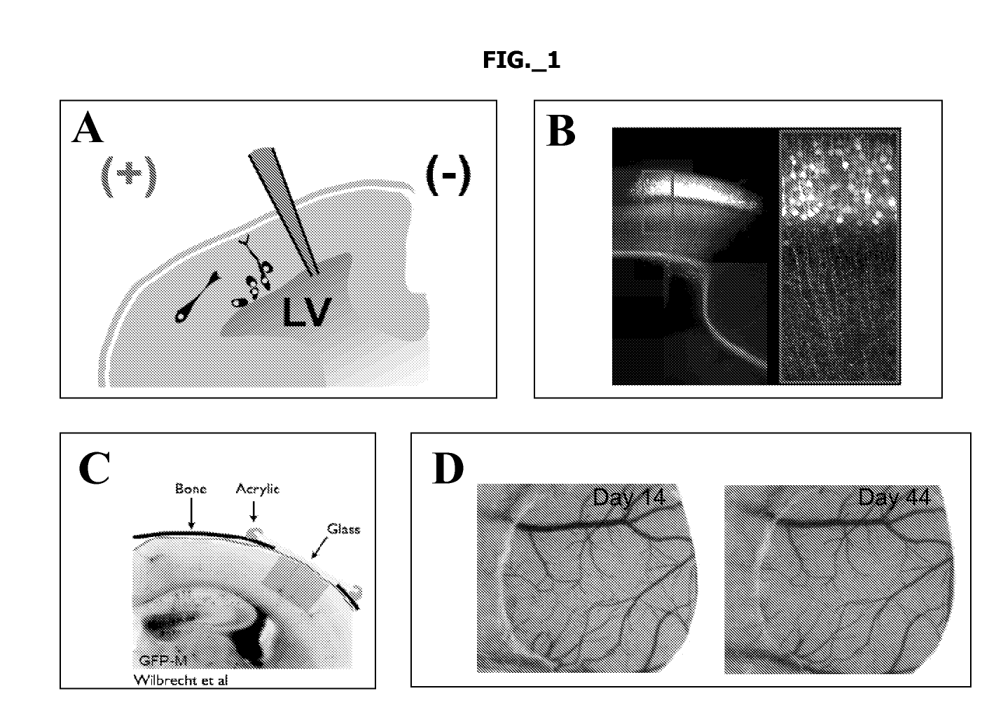

Synapses can be visualized in vivo using 2-photon microscopy through a chronic cranial window (See FIG. 1). Utilizing this approach we can (1) assess density of dendritic spines, which are morphological correlates of excitatory synapses, and (2) morphology of dendritic spines

### Example 1

### Monitoring PSD-95 Retention In Vivo

### Materials and Methods

In utero electroporation. L2/3 progenitor cells were transfected via in utero electroporation (Saito T. and N. Nakatsuji (2001) Dev. Biol. 240:237-246; Tabata, H. and K. Nakajima (2001) Neurosci. 103:865-872). E16 timed-pregnant C57BL/6J mice (Charles River, Wilmington, Mass., United States) were deeply anesthetized using an isoflurane-oxygen mixture. The uterine horns were exposed and approximately 1 μl of DNA solution (containing a plasmid expressing DsRed-Express, 1 ug/ul), and Fast Green [Sigma, St. Louis, Mo., United States]) was pressure injected through a pulled glass capillary tube into the right lateral ventricle of each embryo. The head of each embryo was placed between custom-made tweezer electrodes, with the positive plate contacting the right side of the head. Electroporation was achieved with five square pulses (duration=50 ms, frequency=1 Hz, 40V). Co-transfection efficiencies were 60-70%. See FIG. 1.

Surgery. Imaging windows were installed above the somatosensory cortex at P8 or after P60. Mice were deeply anesthetized with an isoflurane-oxygen mixture. A craniotomy (diameter; 4-5 mm) was opened above the right somatosensory cortex (0.5/1.5 mm posterior from bregma and 3.0/3.5 mm lateral from the midline for pups/adults, respectively), leaving the dura intact. The dura was covered with 1% agarose (Type-IIIA, Sigma) that was dissolved in HEPES-buffered artificial cerebrospinal fluid and covered with a 5 mm custom-made cover glass (No. 1) that was sealed into place with dental acrylic. The animals were also given a 20-μl injection of 4% dexamethasone (Phoenix Scientific, St. Joseph, Mo., United States of America). After a 1-h recovery period, adults were replaced into the cage, and pups were housed with littermates and a surrogate mother.

Imaging. High resolution images were collected via a custom-made two-laser two photon laser scanning microscope (2PLSM). The light source for imaging was a solid-state Ti:Sapphire laser (λ˜1020; ˜100 mW in the objective back-focal plane) (Spectra Physics, Fremont, Calif., United States); Red fluorescence photons were separated using bandpass filters (610/90; Chroma Technology). Signals were collected using photomultiplier tubes (3896; Hamamatsu, Hamamatsu City, Japan). The objective lens (40, 0.8 NA) and trinoc were from Olympus (Tokyo, Japan).

We used the vasculature and the pattern of dendritic branching to identify regions of interest from day to day. Imaging sessions consisted of a series of image stacks over 90 min. Image stacks consisted of individual sections (512×512 pixels; pixel size, 0.08 μm) separated axially by 1 μm. After an imaging session, mice were allowed to recover for approximately 30 min on a warming blanket before housing with littermates and a surrogate mother; adult animals were returned to their cages.

Data analysis. Individual spines were identified, annotated and tracked over timepoints using custom made analysis routines in Matlab (Mathworks). Spine lifetimes, densities, and lengths were measured using custom software (Holtmaat A. J. et al. (2005) Neuron 45:279-291).

### Results

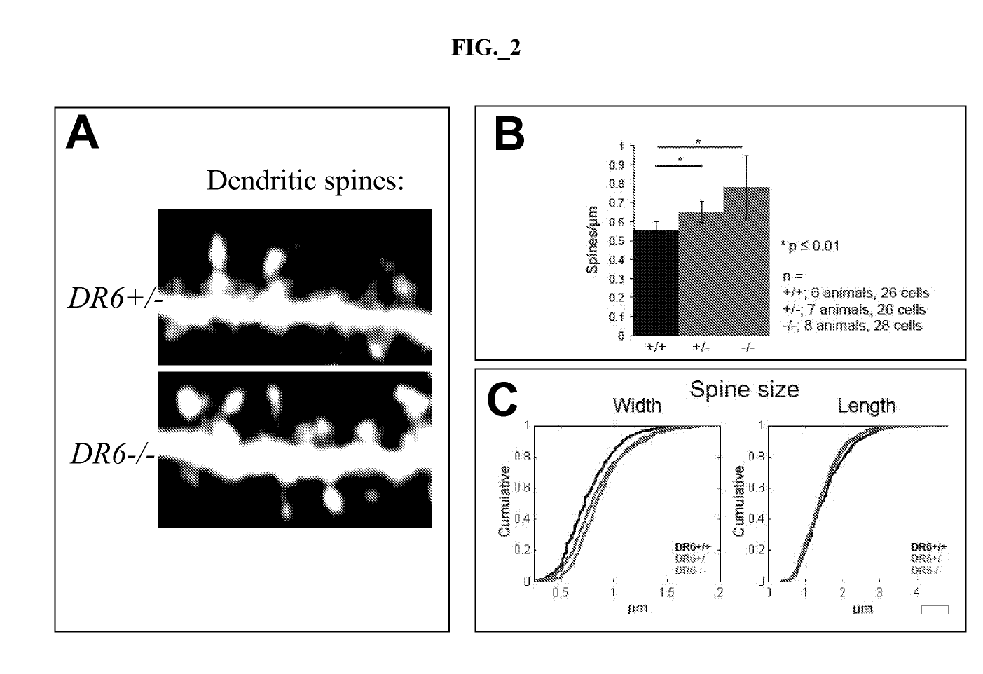

We observed an increased density and width of dendritic spines in DR6^{-/-} animals as compared to DR6^{+/-} and DR6+/+ animals (FIG. 2). Density was calculated by averaging the total number of spines/dendrite length per cell across all animals within the same group. A total of 28 cells/8 animals were annotated for DR6−/− compared to 26 cells/7 animals for DR6+/− and 26 cells/6 animals for DR6+/+. Spine width and length plotted as a cumulative plot of the entire population of spines analyzed per genotype.

The results of Bittner, et al., supra, along with our earlier findings that APP is a cognate ligand for DR6 indicate that APP plays role in dendritic spine density as well.

### Example 2

### Effect of N-APP on Dendritic Spines In Vitro

### Materials and Methods

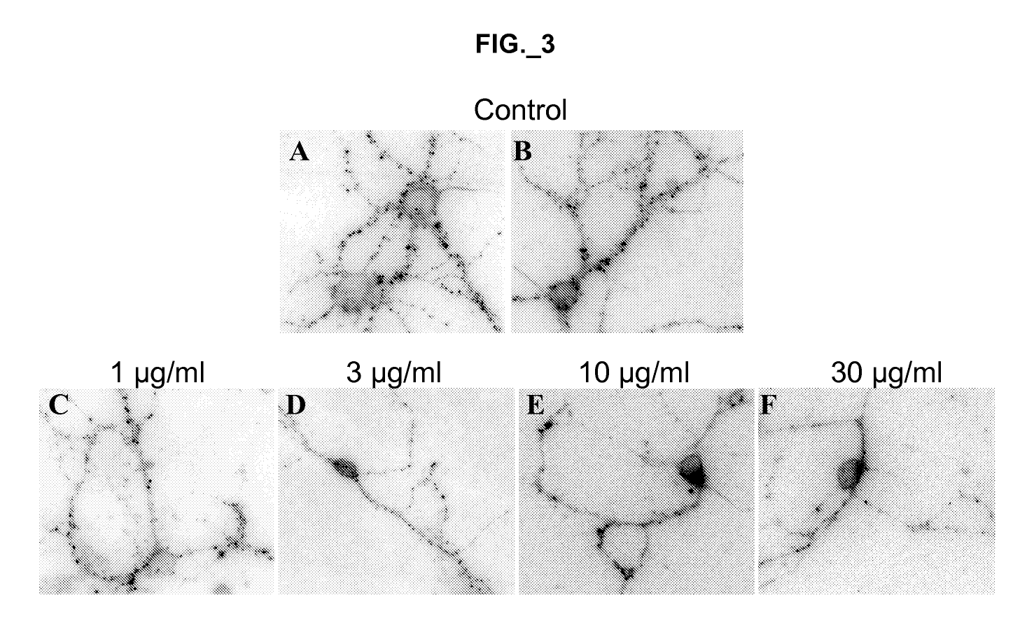

Cell cultures: PDL/Laminin coated 8 well slides (Becton, Dickinson and Company) were filled with 500 μl per well Neurobasal Medium (Invitrogen) plus 50 ng/ml of each recombinant BDNF and NT-3 (Chemicon), plus B-27 supplement X50 (Invitrogen); plus Pen Strep Glutamine X100 (Cat. No. 10378-016; Gibco) plus Glucose X100. E16 cortical neuronal explants were placed in each well and placed in a 37° C. incubator for 21 days. At day 21, cultures were treated with 0, 1, 3, 10, or 30 μg/ml of N-APP. Cultures were incubated for 24 hours. The neurons were then processed for microscopy by fixing and staining using a mouse anti-PSD95 antibody with a secondary antibody (goat-anti-mouse IgG) conjugated to Alexa Fluor® 488 (Molecular Probes). Results are shown in FIG. 3.

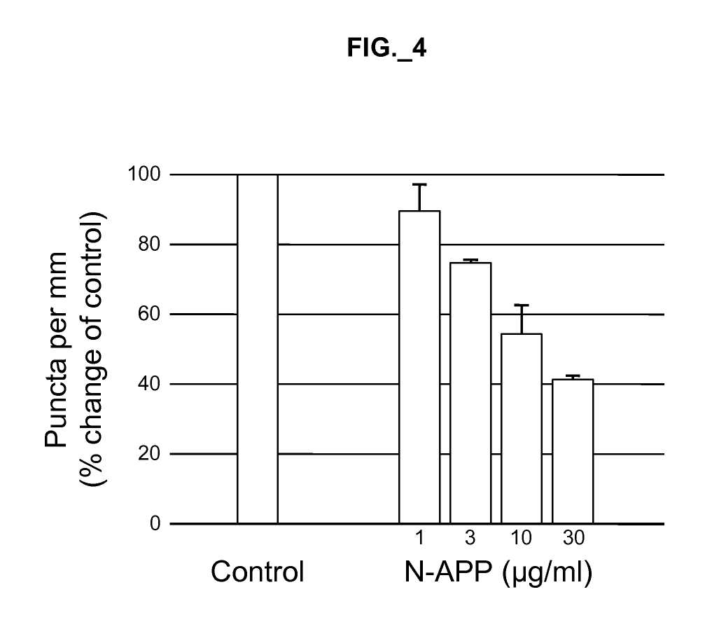

Puncta per mm of neuron were quantified and plotted as a change in puncta/mm as a percent change of untreated cortical neurons (control). The results are shown in FIG. 4.

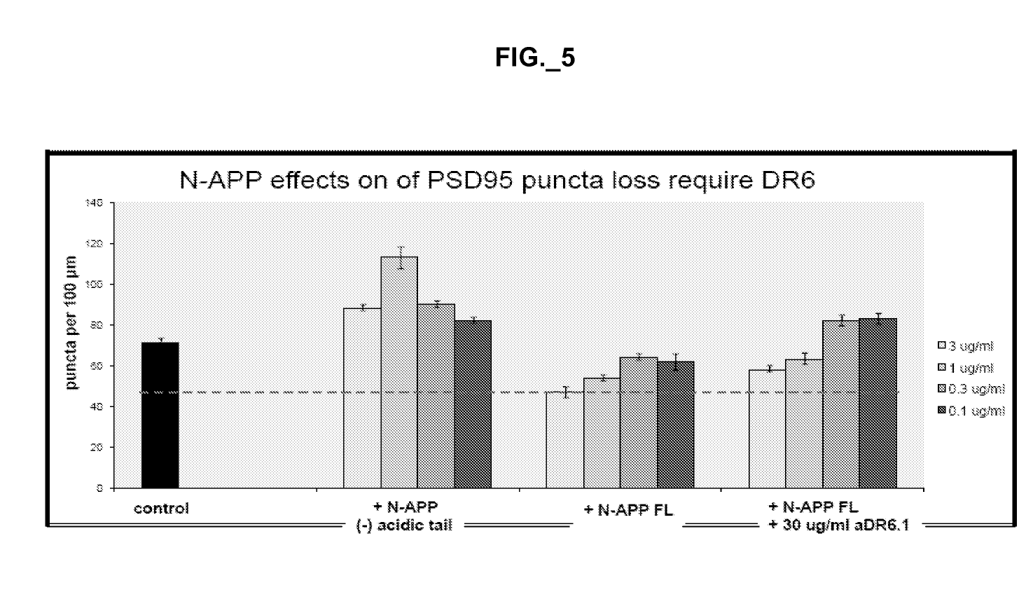

In a separate experiment, cortical cells in culture were exposed to 0 ug/ml N-APP (control) or either N-APP without the acidic tail (N-APP (−) acidic tail), or full-length N-APP (N-APP FL) at concentrations of 0.1, 0.3, 1.0 or 3.0 ug/ml, with or without addition of 30 ug/ml of the anti DR6 antibody aDR6.1. The results are shown in FIG. 5.

### Results

FIG. 3 shows that N-APP triggers a reduction of PSD95 puncta. The reduction in PSD95 puncta was concentration dependent as shown in FIG. 4. The results are consistent with N-APP interacting with DR6 to cause neurodegeneration and/or reduction in neurite (axon or dendrite) length and branching, thus a loss in dentricic spines indicated by the reduction of PSD95 puncta. FIG. 5 shows that the N-APP-induced reduction of PSD95 puncta was dependant on DR6.

What is claimed is:

1. A method of increasing density of dendritic spines in neurons of a patient with a cognitive or psychiatric disorder comprising administering to said patient an effective amount of a DR6 inhibitor or a p75 inhibitor.

2. The method of claim 1 wherein said DR6 inhibitor is an antibody that binds to an epitope of DR6 and inhibits the function of DR6.

3. The method of claim 1 wherein said p75 inhibitor is an antibody that binds to an epitope of p75 and inhibits the function of p75.

4. The method of claim 2 wherein said antibody is selected from the group consisting of 3F4.4.8, 4B6.9.7, 1E5.5.7, and antigen-binding fragments thereof.

5. The method of claim 4 wherein said antibody is a chimeric or humanized 3F4.4.8, 4B6.9.7 or 1E5.5.7, or an antibody that binds to the same epitope as 3F4.4.8, 4B6.9.7 or 1E5.5.7.

6. The method of claim 1 wherein said DR6 inhibitor decreases or prevents DR6 signaling in said neuron.

7. The method of claim 1 wherein said p75 inhibitor decreases or prevents p75 signaling in said neuron.

8. The method of treating a cognitive or psychiatric disorder in a patient in need thereof comprising identifying a patient having a cognitive or psychiatric disorder associated with a decrease in dentritic spines and administering to said patient a therapeutically effective amount of a DR6 antagonist or a p75 antagonist.

9. The method of claim 1 or 8 wherein said psychiatric or cognitive disorder is selected from the group consisting of Rett Syndrome, Tourette syndrome, autism, schizophrenia and fragile-X mental retardation.

10. The method of claim 8 wherein said DR6 inhibitor is an antibody that binds to an epitope of DR6 and inhibits the function of DR6.

11. The method of claim 8 wherein said p75 inhibitor is an antibody that binds to an epitope of p75 and inhibits the function of p75.

12. The method of claim 10 wherein said antibody is selected from the group consisting of 3F4.4.8, 4B6.9.7, 1E5.5.7, and antigen-binding fragments thereof.

13. The method of claim 12 wherein said antibody is a chimeric or humanized 3F4.4.8, 4B6.9.7 or 1E5.5.7, or an antibody that binds to the same epitope as 3F4.4.8, 4B6.9.7 or 1E5.5.7.

14. A method of maintaining cognition in a subject during the aging process comprising administering to said subject an amount of a DR6 inhibitor or a p75 inhibitor effective to promote density of dendritic spines in said subject, thereby maintaining cognition in said subject.

15. The method of claim 14 wherein said DR6 inhibitor is an antibody that binds to an epitope of DR6 and inhibits the function of DR6.

16. The method of claim 15 wherein said antibody is selected from the group consisting of 3F4.4.8, 4B6.9.7, 1E5.5.7, and antigen-binding fragments thereof.

17. The method of claim 16 wherein said antibody is a chimeric or humanized 3F4.4.8, 4B6.9.7 or 1E5.5.7, or an antibody that binds to the same epitope as 3F4.4.8, 4B6.9.7 or 1E5.5.7.

18. The method of claim 14 wherein said p75 inhibitor is an antibody that binds to an epitope of p75 and inhibits the function of p75.

19. Use of a DR6 antagonist in the preparation of a medicament for use in a patient having a cognitive or psychiatric disorder wherein said antagonists inhibits DR6 activity.

20. The use of a DR6 antagonist as recited in claim 15 wherein said DR6 antagonist is an antibody that binds to an epitope of DR6.

21. The use of a DR6 antagonist as recited in claim 16 wherein said antibody is selected from the group consisting of 3F4.4.8, 4B6.9.7, 1E5.5.7, and antigen-binding fragments thereof.

22. The use of a DR6 antagonist as recited in claim 16 wherein said antibody is a chimeric or humanized 3F4.4.8, 4B6.9.7 or 1E5.5.7, or an antibody that binds to the same epitope as 3F4.4.8, 4B6.9.7 or 1E5.5.7.

23. Use of a p75 antagonist in the preparation of a medicament for use in a patient having a cognitive or psychiatric disorder wherein said antagonists inhibits p75 activity.


SEQUENCE LISTING (6 sequences)

SEQ ID NO: 1 - protein, 655 aa, Homo sapiens
MGTSPSSSTALASCSRIARRATATMIAGSLLLLGFLSTTTAQPEQKASNLIGTYRHVDRATGQVLTCDKCPAGTYVSEHCTNTSLRVCSSCPVGTFTRHENGIEKCHDCSQPCPWPMIEKLPCAALTDRECTCPPGMFQSNATCAPHTVCPVGWGVRKKGTETEDVRCKQCARGTFSDVPSSVMKCKAYTDCLSQNLVVIKPGTKETDNVCGTLPSFSSSTSPSPGTAIFPRPEHMETHEVPSSTYVPKGMNSTESNSSASVRPKVLSSIQEGTVPDNTSSARGKEDVNKTLPNLQVVNHQQGPHHRHILKLLPSMEATGGEKSSTPIKGPKRGHPRQNLHKHFDINEHLPWMIVLFLLLVLVVIVVCSIRKSSRTLKKGPRQDPSAIVEKAGLKKSMTPTQNREKWIYYCNGHGIDILKLVAAQVGSQWKDIYQFLCNASEREVAAFSNGYTADHERAYAALQHWTIRGPEASLAQLISALRQHRRNDVVEKIRGLMEDTTQLETDKLALPMSPSPLSPSPIPSPNAKLENSALLTVEPSPQDKNKGFFVDESEPLLRCDSTSSGSSALSRNGSFITKEKKDTVLRQVRLDPCDLQPIFDDMLHFLNPEELRVIEEIPQAEDKLDRLFEIIGVKSQEASQTLLDSVYSHLPDLL

SEQ ID NO: 2 - DNA, 3662 bp, Homo sapiens
gccaccacgtgtgtccctgcgcccggtggccaccgactcagtccctcgccgaccagtctgggcagcggaggagggtggttggcagtggctggaagcttcgctatgggaagttgttcctttgctctctcgcgcccagtcctcctccctggttctcctcagccgctgtcggaggagagcacccggagacgcgggctgcagtcgcggcggcttctccccgcctgggcggccgcgccgctgggcaggtgctgagcgcccctagagcctcccttgccgcctccctcctctgcccggccgcagcagtgcacatggggtgttggaggtagatgggctcccggcccgggaggcggcggtggatgcggcgctgggcagaagcagccgccgattccagctgccccgcgcgccccgggcgcccctgcgagtccccggttcagccatggggacctctccgagcagcagcaccgccctcgcctcctgcagccgcatcgcccgccgagccacagccacgatgatcgcgggctcccttctcctgcttggattccttagcaccaccacagctcagccagaacagaaggcctcgaatctcattggcacataccgccatgttgaccgtgccaccggccaggtgctaacctgtgacaagtgtccagcaggaacctatgtctctgagcattgtaccaacacaagcctgcgcgtctgcagcagttgccctgtggggacctttaccaggcatgagaatggcatagagaaatgccatgactgtagtcagccatgcccatggccaatgattgagaaattaccttgtgctgccttgactgaccgagaatgcacttgcccacctggcatgttccagtctaacgctacctgtgccccccatacggtgtgtcctgtgggttggggtgtgcggaagaaagggacagagactgaggatgtgcggtgtaagcagtgtgctcggggtaccttctcagatgtgccttctagtgtgatgaaatgcaaagcatacacagactgtctgagtcagaacctggtggtgatcaagccggggaccaaggagacagacaacgtctgtggcacactcccgtccttctccagctccacctcaccttcccctggcacagccatctttccacgccctgagcacatggaaacccatgaagtcccttcctccacttatgttcccaaaggcatgaactcaacagaatccaactcttctgcctctgttagaccaaaggtactgagtagcatccaggaagggacagtccctgacaacacaagctcagcaagggggaaggaagacgtgaacaagaccctcccaaaccttcaggtagtcaaccaccagcaaggcccccaccacagacacatcctgaagctgctgccgtccatggaggccactgggggcgagaagtccagcacgcccatcaagggccccaagaggggacatcctagacagaacctacacaagcattttgacatcaatgagcatttgccctggatgattgtgcttttcctgctgctggtgcttgtggtgattgtggtgtgcagtatccggaaaagctcgaggactctgaaaaaggggccccggcaggatcccagtgccattgtggaaaaggcagggctgaagaaatccatgactccaacccagaaccgggagaaatggatctactactgcaatggccatggtatcgatatcctgaagcttgtagcagcccaagtgggaagccagtggaaagatatctatcagtttctttgcaatgccagtgagagggaggttgctgctttctccaatgggtacacagccgaccacgagcgggcctacgcagctctgcagcactggaccatccggggccccgaggccagcctcgcccagctaattagcgccctgcgccagcaccggagaaacgatgttgtggagaagattcgtgggctgatggaagacaccacccagctggaaactgacaaactagctctcccgatgagccccagcccgcttagcccgagccccatccccagccccaacgcgaaacttgagaattccgctctcctgacggtggagccttccccacaggacaagaacaagggcttcttcgtggatgagtcggagccccttctccgctgtgactctacatccagcggctcctccgcgctgagcaggaacggttcctttattaccaaagaaaagaaggacacagtgttgcggcaggtacgcctggacccctgtgacttgcagcctatctttgatgacatgctccactttctaaatcctgaggagctgcgggtgattgaagagattccccaggctgaggacaaactagaccggctattcgaaattattggagtcaagagccaggaagccagccagaccctcctggactctgtttatagccatcttcctgacctgctgtagaacatagggatactgcattctggaaattactcaatttagtggcagggtggttttttaattttcttctgtttctgatttttgttgtttggggtgtgtgtgtgtgtttgtgtgtgtgtgtgtgtgtgtgtgtgtgtgtgtgtttaacagagaatatggccagtgcttgagttctttctccttctctctctctcttttttttttaaataactcttctgggaagttggtttataagcctttgccaggtgtaactgttgtgaaatacccaccactaaagttttttaagttccatattttctccattttgccttcttatgtattttcaagattattctgtgcactttaaatttacttaacttaccataaatgcagtgtgacttttcccacacactggattgtgaggctcttaacttcttaaaagtataatggcatcttgtgaatcctataagcagtctttatgtctcttaacattcacacctactttttaaaaacaaatattattactatttttattattgtttgtcctttataaattttcttaaagattaagaaaatttaagaccccattgagttactgtaatgcaattcaactttgagttatcttttaaatatgtcttgtatagttcatattcatggctgaaacttgaccacactattgctgattgtatggttttcacctggacaccgtgtagaatgcttgattacttgtactcttcttatgctaatatgctctgggctggagaaatgaaatcctcaagccatcaggatttgctatttaagtggcttgacaactgggccaccaaagaacttgaacttcaccttttaggatttgagctgttctggaacacattgctgcactttggaaagtcaaaatcaagtgccagtggcgccctttccatagagaatttgcccagctttgctttaaaagatgtcttgttttttatatacacataatcaataggtccaatctgctctcaaggccttggtcctggtgggattccttcaccaattactttaattaaaaatggctgcaactgtaagaacccttgtctgatatatttgcaactatgctcccatttacaaatgtaccttctaatgctcagttgccaggttccaatgcaaaggtggcgtggactccctttgtgtgggtggggtttgtgggtagtggtgaaggaccgatatcagaaaaatgccttcaagtgtactaatttattaataaacattaggtgtttgttaaaaaaaaaaaaaaaaaaaaaaaaaaaaa

SEQ ID NO: 3 - protein, 695 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVRVPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 4 - protein, 751 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAIPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 5 - protein, 770 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 6 - protein, 427 aa, Homo sapiens
MGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNSCKQNKQGANSRPVNQTPPPEGEKLHSDSGISVDSQSLHDQQPHTQTASGQALKGDGGLYSSLPPAKREEVEKLLNGSAGDTWRHLAGELGYQPEHIDSFTHEACPVRALLASWATQDSATLDALLAALRRIQRADLVESLCSESTATSPV